(19) United States
(12) Patent Application Publication
(10) Pub. No.: US 2008/0124270 A1
(43) Pub. Date: May 29, 2008

(54) Compounds Useful as Metal Chelators

(75) Inventors: Michael F. Tweedle, Princeton, NJ (US); Hong Fan, Plainsboro, NJ (US); Luciano Lattuada, Bussero (IT); Kondareddiar Ramalingam, Dayton, NJ (US); Rolf E. Swenson, Princeton, NJ (US)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: BRACCO IMAGING S.P.A., MILAN ITALY (IT)

(21) Appl. No.: 10/584,430

(22) PCT Filed: Dec. 20, 2004
(86) PCT No.: PCT/US04/42710

§ 371 Date: Jun. 22, 2006

Related U.S. Application Data
(60) Provisional application No. 60/532,842, filed on Dec. 23, 2003.

(51) Int. Cl. A61K 51/00; C07D 255/02; A61P 43/00; A61B 5/055

(52) U.S. Cl. 424/1.11; 540/474; 424/9.363

(57) ABSTRACT

New compounds are provided that may be used to chelute a metal. The compound comprise polyazamacrocyclic compound with at least one phosphonic group substituted on at least one of the aza groups of the polyazamacrocyclic compound. Methods for preparing the compounds are also provided. Methods for preparing a diagnostic imaging agent using the compounds and methods for diagnostic imaging are further provided. Methods for preparing a therapeutic agent using the compounds and methods for therapy are further provided.

### CROSS REFERENCE TO RELATED APPLICATIONS

This application claims the benefit of and priority to U.S. Provisional Application No. 60/532,842, filed Dec. 23, 2003, hereby incorporated by reference in its entirety.

### FIELD OF THE INVENTION

This invention relates to new compounds that are capable of being complexed with a metal to form diagnostic or therapeutic agents. The compounds can also be attached or linked to a targeting moiety or a diagnostic or therapeutic moiety before or after complexation with a metal to provide targeted imaging or treatment.

### BACKGROUND OF THE INVENTION

Pharmaceutical agents (e.g., as diagnostic imaging agents, therapeutic agents) have been used to enhance images of body and organ morphology and structure and/or to detect and treat disease. These pharmaceutical agents may comprise a compound that is capable of forming a chelation complex with a metal, such as a paramagnetic or lanthanide metal, that is useful in diagnostic imaging and/or the treatment of disease. Without the formation of such a complex, the metal may be too toxic to use or it may have unfavorable distribution, metabolism or elimination properties or some other undesirable effect in an animal or human. Thus, compounds are needed that help reduce the toxicity of the metal and/or aid in the distribution, metabolism or elimination of the metal. Compounds that bind to a targeting moiety that directs the resulting agent to a particular site or metabolic function, thereby permitting the imaging of specific organs or structures and disease detection and/or treatment are also needed.

Regarding magnetic resonance imaging (“MRI”) in particular, compounds that chelate gadolinium are used in clinical practice as agents that enhance images. For example, the Gd(III) complex of diethylene triamine pentaacetic acid, the Gd(III) complex of 1,4,7,10-tetraazacyclododecane-1,4,7,10-tetraacetic acid, the Gd(III) complex of 10-(2-hydroxypropyl)-1,4,7,10-tetraazacyclododecane-1,4,7,-tri acetic acid, and the Gd (III) complex of diethylenetriamine pentaacetic acid bis(methylamide) are used in clinical practice as MRI agents. Many of the polyaza ligands described in the literature are macrocycles that have identical coordinating pendent arms on the nitrogen atoms. It has been observed that selective functionalization of the polyazamacrocycle presents a significant synthetic challenge.

One measure of a complex of a paramagnetic metal and a chelation compound is its signal enhancing effect when used as a contrast agent for MRI. The effectiveness of a paramagnetic complex (e.g., a complex including Gd (III)) in decreasing the relaxation time (T₁) of the vicinal water protons is an intrinsic property called relaxivity, r₁, which is given by the following equation:

r₁=BQτ_{c}/A⁶

where B is a constant, A is the proton to Gd distance, Q is the number of coordinated water molecules that are exchanging with bulk water, and τ_{c} the overall correlation time. It is evident from the equation that the relaxivity will increase with increasing Q and τ_{c}. The correlation time, τ_{c}, is a combination of the rotational correlation time, τᵣ, the electronic relaxation time, τₛ, and the exchange time of the coordinated water molecule on the metal, τₘ, and is given by the equation shown below:

τ_{c}⁻¹=τᵣ⁻¹⁺τₛ⁻¹⁺τₘ⁻¹

High relaxation rates ensure increased contrast in the image. Increased contrast makes it possible to obtain better physiological information in a shorter period of time, which has significant advantages in terms of image quality and cost. Compounds with enhanced relaxivity provide a stronger signal enhancing effect per molecule than can be obtained with more typical relaxation agents that are used in contrast enhancement. One application that could take advantage of a high relaxivity chelate is to link a compound or complex to a bioactive compound or targeting moiety that targets a particular tissue. Localization at the target via the targeting moiety would result in a higher signal enhancement than can be obtained if a comparable compound were linked that had normal relaxivity. Therefore, synthesis of high relaxivity compounds that are capable of being attached or linked to targeting moieties can be a worthy goal.

Compounds also can be used to form chelation complexes with lanthanides and radionuclides. Compounds that reduce the toxicity of the lanthanide or radionuclide when they are used as contrast agents or radiopharmaceuticals and/or that improve the distribution, metabolism or elimination of the metal, whether because they are bound to a targeting moiety or otherwise, would be beneficial.

### SUMMARY OF THE INVENTION

In an exemplary embodiment of the present invention, there is provided new compounds that are capable of being metal chelators (e.g., chemical moieties capable of complexing a medically useful metal ion or radionuclide). The compounds complexed to a metal may be useful for diagnostic imaging and/or the treatment of disease.

The new compounds of this invention comprise polyazamacrocycle compounds (Aza) comprising at least one phosphonic acid group (P), where n=1 to 12:

Aza-Pₙ (I)

These new compounds may be capable of chelating with bi-valent and tri-valent metal ions. Complexes formed from the new compound and a metal may serve as contrast agents for MRI or as a radiopharmaceutical for radionuclide (e.g., scintigraphic) imaging. The new compound and an appropriate metal may also serve as therapeutic agents for the treatment of diseases such as cancer.

Exemplary polyazamacrocyclic compounds of this invention may be depicted by the general formula (II):

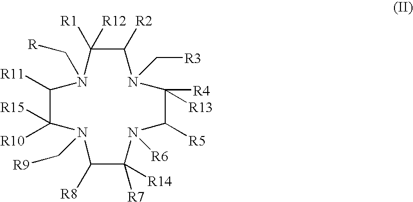

where

### R₁═R²═R⁷═R⁸═R¹⁰═R¹¹═H;

### R¹², R¹³, R¹⁴, and R¹⁵═CH₃ or H;

R⁴═R⁵ and R¹⁰═R¹¹ can be H or groups taken together forming a cyclic C₃-C₄ alkene group; at least one of R, R³, R⁶ or R⁹═X, where X═CH₂P(O) (OH)₂, CH₂P(O) (OC₄H₉-t)₂, CH₃CHP(O) (OH)₂, CHP(O) (OH)₂—(CH₂)ₙCO₂H, CHP(O) (OH)₂, (CH₂)ₙNH₂, CHP(O) (OH)₂-Aryl-CO₂H, CHP(O) (OH)₂-Aryl-NH₂ or CHP(O) (OH)₂-Aryl-NHCS and n=1-12; and
when R, R³, R⁶ or R⁹ are not X, then that R, R³, R⁶ or R⁹ is CO₂C(CH)₃, or CO₂H.

Further preferred and exemplary polyazamacrocyclic compounds of this invention may be depicted by the general formula (III):

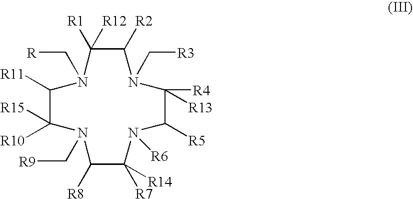

where

### R═R³═R⁹═CO₂C(CH)₃, or CO₂H;

### R¹═R²═R⁴═R⁵═R⁷═R⁸═R¹⁰═R¹¹═H;

### R¹², R¹³, R¹⁴, and R¹⁵═CH₃ or H;

### R¹⁰═R¹¹ can be H or groups taken together forming a cyclic C₃-C₄ alkene group; and

### R⁶═CH₂P(O) (OH)₂, CH₂P(O) (OC₄H₉-t)₂, CH₃CHP(O) (OH)₂, CHP(O) (OH)₂—(CH₂)ₙCO₂H, CHP(O) (OH)₂, (CH₂)ₙNH₂, CHP(O) (OH)₂-Aryl-CO₂H or CHP(O) (OH)₂-Aryl-NH₂, where n=1-12.

Further preferred and exemplary polyazamacrocyclic compounds of this invention may be depicted by the general formula (IV):

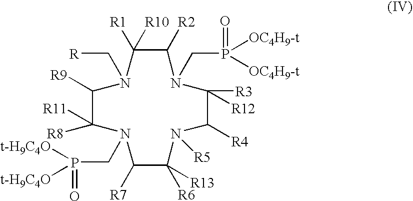

where

### R¹═R²═R³═R⁴═R⁶═R⁷═R⁸═R⁹═H;

### R³═R⁴ and R⁸═R⁹ can be H or groups taken together forming a cyclic C₃-C₄ alkene group;

### R¹⁰, R¹¹, R¹² and R¹³═CH₃ or H;

### R═CO₂C(CH₃)₃; and

### R⁵═CH₂P(O) (OH)₂, CH₂P(O) (OC₄H₉-t)₂, CH₃CHP(O) (OH)₂, CHP(O) (OH)₂—(CH₂)ₙCO₂H, CHP(O) (OH)₂, (CH₂)ₙNH₂, CHP(O) (OH)₂-Aryl-CO₂H, CHP(O) (OH)₂-Aryl-NH₂ or CHP(O) (OH)₂-Aryl-NHCS, where n=1-12.

The compounds of this invention may also be combined to form homo and hetero dimers and homo and hetero multimers.

The compounds of this invention may or may not be complexed with a metal such as a radionuclide, paramagnetic metal or a lanthanide.

The compounds and complexes of this invention may also be in the form of salts. Preferred cations of inorganic bases that can be suitably used to salify the complexes of the invention comprise alkali or alkaline earth metals such as sodium, potassium, calcium and magnesium, among others. Preferred cations of organic bases are N-methyl glucamine and N,N-dimethyl glucamine and diethanolamine.

An optional linker may be bound to the compound or the complex of a compound and a metal and may comprise a chemical bond, a chemical group, a peptide or some other chemical entity.

An optional targeting moiety, which is any chemical entity, such as a peptide, hormone, bile acid, protein, oligonucleotide, antibody, antigen or other chemical entity or equivalents, derivatives or analogs of the foregoing, which has binding affinity for a particular site or metabolic function, may also be used. The targeting moiety may be bound to a linking group that is attached to the compound or a complex of the compound and a metal. Alternatively, the targeting moiety may be directly bound to the compound or a complex of the compound and a metal. The targeting moiety is preferably a peptide that binds to a receptor or enzyme of interest. For example, the targeting peptide may be LHRH, insulin, oxytocin, somatostatin, NK-1, VIP, GRP, bombesin or any other hormone peptides known in the art, as well as analogs and derivatives thereof. Additionally, other diagnostic or therapeutic moieties may be attached to the chelators of the invention, either directly or indirectly via a linker.

Methods for preparing the metal chelators and using them to prepare diagnostic imaging agents and therapeutic agents are disclosed in more detail below. Such imaging agents may be prepared by a method comprising the step of adding to an injectable imaging medium a substance containing the metal chelating compounds of the present invention. Such therapeutic agents may be prepared by a method comprising the step of adding to an injectable therapeutic medium a substance comprising a compound of the invention. These diagnostic and therapeutic agents may also be made available in kits that aid in their preparation and use.

When used as MRI agents, complexes of the invention may exhibit immobilized relaxivity in the range of 30-200 mM⁻¹s⁻¹, although greater or lesser ranges can be potentially achieved.

This invention also includes a novel method of imaging and a novel method of radiotherapy using the compounds of the present invention.

Further descriptions of this invention and embodiments continue below.

### DETAILED DESCRIPTION OF THE INVENTION

In the following description, various aspects of the present invention will be further elaborated. For purposes of explanation, specific configurations and details are set forth in order to provide a thorough understanding of the present invention. However, it will also be apparent to one skilled in the art that the present invention may be practiced without the specific details. Furthermore, well known features may be omitted or simplified in order not to obscure the present invention.

### Metal Chelating Compounds, Precursors and Metal Complexes

The term “metal chelator” refers to a compound that is capable of forming a complex with a metal atom, wherein the complex is relatively stable under physiological conditions. That is, the metal will remain complexed to a significant extent to the chelator in vivo. More particularly, a metal chelator is a molecule that is capable of complexing to a paramagnetic, lanthanide or other radionuclide metal to form a metal complex that is relatively stable under physiological conditions. The metal chelating compound may or may not be complexed with a metal or a radionuclide.

The present invention relates to new compounds that are capable of being metal chelators comprising polyazamacrocycle compounds (Aza) comprising at least one phosphonic acid group (P), where n=1-12:

Aza-Pₙ (I)

The phosphonic acid group may act as a coordinating pendent arm on a nitrogen of the compound if the compound is used to chelate a metal ion. These new compounds may be capable of chelating bi-valent and tri-valent metal ions.

Exemplary polyazamacrocyclic compounds of this invention may be depicted by the general formula (II):

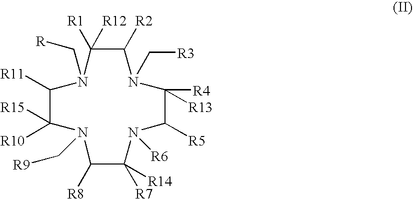

where

### R¹═R²═R⁷═R⁸═R¹⁰═R¹¹═H;

### R¹², R¹³, R¹⁴, and R¹⁵═CH₃ or H;

R⁴═R⁵ and R¹⁰═R¹¹ can be H or groups taken together forming a cyclic C₃-C₄ alkene group; at least one of R, R³, R⁶ or R⁹═X, where X═CH₂P(O) (OH)₂, CH₂P(O) (OC₄H₉-t)₂, CH₃CHP(O) (OH)₂, CHP(O) (OH)₂—(CH₂)ₙCO₂H, CHP(O) (OH)₂, (CH₂)ₙNH₂, CHP(O) (OH)₂-Aryl-CO₂H, CHP(O) (OH)₂-Aryl-NH₂ or CHP(O) (OH)₂-Aryl-NHCS, where n=1-12; and
when R, R³, R⁶ or R⁹ are not X, then that R, R³, R⁶ or R⁹ is CO₂C(CH)₃, or CO₂H.

Further preferred and exemplary Polyazamacrocyclic compounds of this invention may be depicted by the general formula (III):

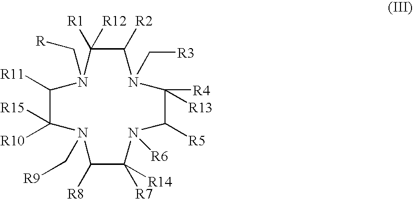

where

### R═R³═R⁹═CO₂C(CH)₃, or CO₂H;

### R¹═R²═R⁴═R⁵═R⁷═R⁸═R¹⁰═R¹¹═H;

### R¹², R¹³, R¹⁴, and R¹⁵═CH₃ or H;

### R¹⁰═R¹¹ can be H or groups taken together forming a cyclic C₃-C₄ alkene group; and

### R⁶═CH₂P(O) (OH)₂, CH₂P(O) (OC₄H₉-t)₂, CH₃CHP(O) (OH)₂, CHP(O) (OH)₂—(CH₂)ₙCO₂H, CHP(O) (OH)₂, (CH₂)ₙNH₂, CHP(O) (OH)₂-Aryl-CO₂H or CHP(O) (OH)₂-Aryl-NH₂, where n=1-12.

Further preferred and exemplary compounds of this invention may be depicted by the general formula (IV):

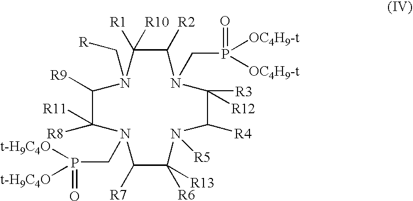

where

### R¹═R²═R³═R⁴═R⁶═R⁷═R⁸R⁹═H;

### R³═R⁴ and R⁸═R⁹ can be H or groups taken together forming a cyclic C₃-C₄ alkene group;

### R¹⁰, R¹¹, R¹² and R¹³═CH₃ or H;

### R═CO₂C(CH₃)₃; and

### R⁵═CH₂P(O) (OH)₂, CH₂P(O) (OC₄H₉-t)₂, CH₃CHP(O) (OH)₂, CHP(O) (OH)₂—(CH₂)ₙCO₂H, CHP(O) (OH)₂, (CH₂)ₙNH₂, CHP(O) (OH)₂-Aryl-CO₂H, CHP(O) (OH)₂-Aryl-NH₂ or CHP(O) (OH)₂-Aryl-NHCS, where n=1-12.

The compounds of this invention may also be combined to form homo and hetero dimers and homo and hetero multimers.

Other compounds of this invention include compounds of formulas (I), (II), (III) and (IV) wherein the phosphonic and/or the carboxylic acid groups are protected as t-butyl esters so that deprotection can be effected in one step under mild conditions (i.e., TFA cleavage compatible with peptide synthesis). Facile removal of the protecting groups makes the compounds of this invention extremely useful synthons in combinatorial library synthesis.

Certain compounds of this invention are capable of being conjugated with suitable molecules able to interact with physiological systems, e.g., targeting moieties. Useful examples of targeting moieties are peptides, hormones, bile acids, proteins, oligonucleotides, antibodies, antigens or other chemical entities and equivalents, derivatives or analogs of the foregoing (described further below). Such compounds will preferrably contain at least one functional group that is capable of conjugation with the suitable molecules.

Furthermore, the metal chelating compound can include an optional spacer such as a chemical entity such as a chemical group or one or more amino acids (e.g., Gly), which does not significantly complex with the metal, but which creates a physical separation between the metal chelator and the linker, targeting moiety, etc.

The metal chelating compound may be combined with a metal to form a chelated complex of the compound and the metal. As examples, the invention further relates to chelates of compounds of formula (I), (II), (III) and (IV) with paramagnetic or radioactive metal ions in particular with bivalent or trivalent ions of the elements having the atomic number ranging between 20 and 31, 39, 42, 43, 44, 49 and between 57 and 83, as well as salts thereof with physiologically compatible bases and acids.

For uses as diagnostic agents using MRI, the compounds are preferably complexed with paramagnetic ions such as Gd³⁺, Dy³⁺, Fe³⁺, Fe²⁺ and Mn²⁺. Particularly preferred is Gd³⁺. For uses as diagnostic agents using radionuclide (e.g., scintigraphic imaging) or in radiotherapy, the compounds are preferably complexed with ¹¹¹In, ⁶²Cu, ¹⁵³Sm and ¹⁷⁷Lu ⁹⁰Y, ¹⁶⁶Ho or ¹¹¹In. Particularly preferred are ¹⁷⁷Lu and ¹¹¹In. When used as MRI agents, complexes of the invention may exhibit immobilized relaxivity in the range of 30-200 mM⁻¹s⁻¹, although greater or lesser ranges can be potentially achieved.

Further preferred groups of metal radionuclides for scintigraphy or radiotherapy include ^{99m}Tc, ^{|}Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵ Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ¹⁹⁸Au and ¹⁹⁹Au and oxides or nitrides thereof. The choice of metal will be determined based on the desired therapeutic or diagnostic application. For example, for diagnostic purposes (e.g., to diagnose and monitor therapeutic progress in treating diseases, including primary tumors and metastases), the preferred radionuclides include ⁶⁴Cu, ⁶⁷Ga, ⁶⁸Ga, and ¹¹¹In, with ¹¹¹In being especially preferred. For therapeutic purposes (e.g., to provide radiotherapy for diseases including primary tumors and metastasis related to cancers of the prostate, breast, lung, etc.), the preferred radionuclides include ⁶⁴Cu, ⁹⁰Y, ¹⁰⁵Rh, ¹¹¹In, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, and ¹⁹⁹Au, with ¹⁷⁷Lu, ⁹⁰Y, being particularly preferred. Compounds labeled with ¹⁷⁷Lu, ⁹⁰Y or other therapeutic radionuclides can be used to provide radiotherapy for primary tumors and metastases related to cancers of the prostate, breast, lung, etc.

The compounds and complexes of this invention may also be in the form of salts. Compounds and complexes with salifiable functional groups are particular examples that may be the form of salts. Preferred cations of inorganic bases that can be suitably used to salify the complexes of the invention comprise alkali or alkaline earth metals such as sodium, potassium, calcium and magnesium, among others. Preferred cations of organic bases are n-methyl glucamine and n, n-dimethyl glucamine and diethanolamine.

### Other Diagnostic Moieties

In alternative embodiments, compounds of the present invention can incorporate other diagnostic moieties, such as agents that enable detection of the compounds by such techniques as x-ray, MRI, ultrasound, fluorescence and other optical imaging methodologies and other techniques that are used, being developed or that will be developed. The choice of diagnostic moiety will be determined based on the desired application.

### Other Therapeutic Moieties

In alternative embodiments, compounds of the present invention can incorporate other therapeutic moieties such as antibiotics, hormones, enzymes, antibodies, growth factors and other such moieties that are used, being developed or that will be developed. The choice of therapeutic moiety will be determined based on the desired application.

Alternatively, compounds of the invention may be administered in combination with a therapeutic moiety. The choice of such therapeutic moieties will also be determined based on the desired application. Suitable therapeutic moieties include, but are not limited to: antineoplastic agents, such as, for example, platinum compounds (e.g., spiroplatin, cisplatin, and carboplatin), methotrexate, adriamycin, mitomycin, ansamitocin, bleomycin, cytosine, arabinoside, arabinosyl adenine, mercaptopolylysine, vincristine, busulfan, chlorambucil, melphalan (e.g., PAM, a, L-PAM or phenylalanine mustard), mercaptopurine, mitotane, procarbazine hydrochloride, dactinomycin (actinomycin D), daunorubicin hydrochloride, doxorubicin hydrochloride, taxol, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase), Erwina aparaginase, etoposide (VP-16), interferon α-2a, interferon α-2b, teniposide (VM-26), vinblastine sulfate (VLB), and arabinosyl; blood products such as, for example, parenteral iron, hemin, hematoporphyrins and their derivatives; biological response modifiers such as, for example, muramyldipeptide, muramyltripeptide; microbial cell wall components; lymphokines (e.g., bacterial endotoxin such as lipopolysaccharide, macrophage activation factor); sub-units of bacteria (such as Mycobacteria, Corynebacteria); the synthetic dipeptide N-acetyl-muramyl-1-alanyl-I)-isoglutamine; anti-fungal agents; hormones; vitamins; retinoids and derivatives; enzymes; anti-allergic agents; anti-coagulation agents; circulatory drugs; metabolic potentiators such as, for example, glutathione; antituberculars; antivirals; antianginals; antibiotics, anti-inflammatories; antiprotozoans; antirheumatics; narcotics; opiates; cardiac glycosides; neuromuscular blockers; sedatives (hypnotics); local anesthetics; and general anesthetics. In certain embodiments, the therapeutic may be a monoclonal antibody or a fragment thereof, such as a monoclonal antibody capable of binding to melanoma antigen or another tumor or cancer marker.

### Linker or Linking Groups

Optional linking groups (also called linkers) may be used with the compounds and complexes of this invention. Such linkers can include a chemical bond, a chemical group or a compound that serves to couple a targeting moiety to the metal chelator. It is preferred that the linker not significantly adversely affect either the targeting function of the targeting moiety or the metal complexing function of the metal chelator. Suitable linkers include chemical bonds, peptides (i.e., amino acids linked together) alone, a non-peptide group (e.g., hydrocarbon chain), a combination of an amino acid sequence and a non-peptide group, or any other chemical entity that achieves the desired result of linking a targeting moiety to a compound or complex of this invention. Several specific linking groups are described below as examples.

In one embodiment, linking groups include substituted bile acids and L-glutamine or hydrocarbon chains, or a combination thereof.

In another embodiment, linking groups include substituted bile acids and a pure peptide linking group consisting of a series of amino acids (e.g., diglycine, triglycine, gly-gly-glu, gly-ser-gly).

In yet a further embodiment, linking groups can also include substituted bile acids and a hydrocarbon chain [i.e., R₁—(CH₂)ₙ—R₂] wherein n is 0-10, preferably n=3 to 9, R₁ is a group (e.g., H₂N—, HS—, —COOH) that can be used as a site for covalently linking the ligand backbone or the preformed metal chelator; and R₂ is a group that is used for covalent coupling to the N-terminal NH₂-group of a given targeting peptide (e.g., R₂ is an activated COOH group). Several chemical methods for conjugating ligands (i.e., chelators) or chelates (chelators/ligands complexed with a radionuclide) to biomolecules (such as targeting peptides) have been well described in the literature (Wilbur, 1992; Parker, 1990; Hermanson, 1996; Frizberg et al., 1995). One or more of these methods could be used to couple either the uncomplexed ligand (chelator) or the metal chelate to the linker or to couple the linker to the targeting moiety. These methods include the formation of acid anhydrides, aldehydes, arylisothiocyanates, activated esters, or N-hydroxysuccinimide esters (Wilbur, 1992; Parker, 1990; Hermanson, 1996; Frizberg et al., 1995).

In a preferred embodiment, other linkers may be formed in whole or in part from linker precursors having electrophiles or nucleophiles as set forth below:

LP1: a linker precursor having on at least two locations of the linker the same electrophile E1 or the same nucleophile Nu1;

LP2: a linker precursor having an electrophile E1 and on another location of the linker a different electrophile E2;

LP3: a linker precursor having a nucleophile Nu1 and on another location of the linker a different nucleophile Nu2; or

LP4: a linker precursor having one end functionalized with an electrophile E1 and the other with a nucleophile Nu1.

The preferred nucleophiles Nu1/Nu2 include —OH, —NH, —NR, —SH, —HN—NH₂, —RN—NH₂, and —RN—NHR′, in which R′ and R are independently selected from the definitions for R given above, but for R′ is not H.

The preferred electrophiles E1/E2 include —COOH, —CH═O (aldehyde), —CR═OR′ (ketone), —RN—C═S, —RN—C═O, —S—S-2-pyridyl, —SO₂—Y, —CH₂C(═O)Y, and

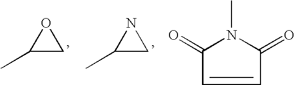

wherein Y can be selected from the following groups:

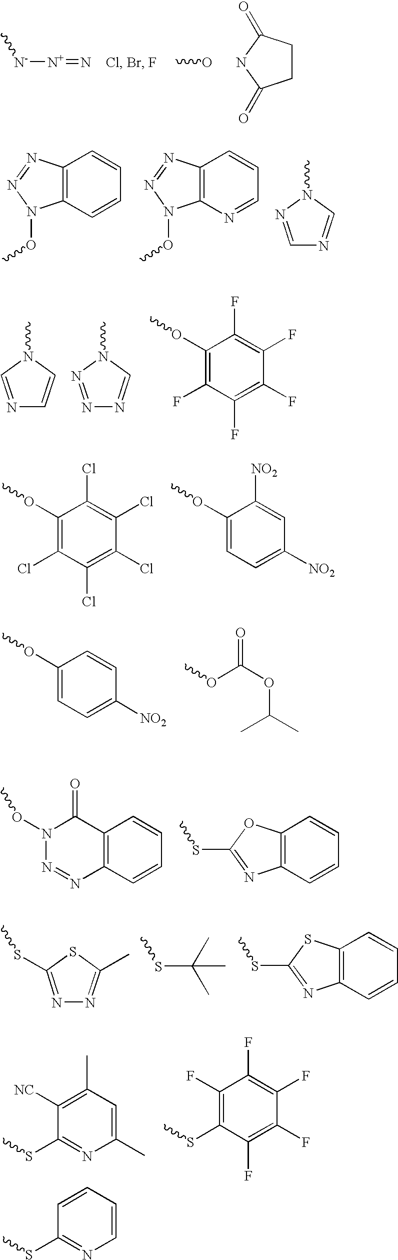

Particularly preferred linkers include those disclosed in co-pending applications U.S. Ser. No. 60/439,722 and PCT/US03/41656, which applications are hereby incorporated by reference in their entirety.

### Targeting Moieties

The targeting moiety is any molecule that has a binding affinity for a particular site or a specific metabolic function. The targeting moiety directs the compounds of the invention to the appropriate site, or involves the compounds in a reaction, where the desired diagnostic or therapeutic activity will occur. In an exemplary embodiment, the targeting moiety may be a peptide, equivalent, derivative or analog thereof, which functions as a ligand that binds to a particular site. In another exemplary embodiment, the targeting moiety may be an enzyme, or a molecule that binds to an enzyme. In another exemplary embodiment, the targeting moiety may be an antibiotic.

In a preferred embodiment, the targeting moiety is a peptide that binds to a receptor or enzyme of interest. For example, a targeting moiety may be a peptide hormone such as, for example, leutinizing hormone releasing hormone (LHRH) such as that described in the literature (e.g., Radiometal-Binding Analogs of Leutenizing Hormone Releasing Hormone PCT/US96/08695; PCT/US97/12084 (WO 98/02192)); insulin; oxytocin; somatostatin; Neurokinin-1 (NK-1); Vasoactive Intestinal Peptide (VIP) including both linear and cyclic versions as delineated in the literature, (e.g., Comparison of Cyclic and Linear Analogs of Vasoactive Intestinal Peptide. D. R. Bolin, J. M. Cottrell, R. Garippa, N. Rinaldi, R. Senda, B. Simkio, M. O'Donnell. Peptides: Chemistry, Structure and Biology Pravin T. P. Kaumaya, and Roberts S. Hodges (Eds). Mayflower Scientific LTD., 1996, pgs 174-175); gastrin releasing peptide (GRP); bombesin and other known hormone peptides, as well as analogs and derivatives thereof.

Other useful targeting moieties include analogs of somatostatin, which, for example, are Lanreotide (Nal-Cys-Thr-DTrp-Lys-Val-Cys-Thr-NH₂), Octreotide (Nal-Cys-Thr-DTrp-Lys-Val-Cys-Thr-ol), and Y³-Octreotate (DPhe-Cys-Tyr-DTrp-Lys-Thr-Cys-Thr-OH). These analogs are described in the literature (e.g., Potent Somatostatin Analogs Containing N-terminal Modifications, S. H. Kim, J. Z. Dong, T. D. Gordon, H. L. Kimball, S. C. Moreau, J.-P. Moreau, B. A. Morgan, W. A. Murphy and J. E. Taylor; Peptides: Chemistry, Structure and Biology Pravin T. P. Kaumaya, and Roberts S. Hodges (Eds), Mayflower Scientific LTD., 1996, pgs 241-243.)

Still other useful targeting moieties include Substance P agonists (e.g., G. Bitan, G. Byk, Y. Mahriki, M. Hanani, D. Halle, Z. Selinger, C. Gilon, Peptides: Chemistry, Structure and Biology, Pravin T. P. Kaumaya, and Roberts S. Hodges (Eds), Mayflower Scientific LTD., 1996, pgs 697-698; G Protein Antagonists A novel hydrophobic peptide competes with receptor for G protein binding, Hidehito Mukai, Eisuke Munekata, Tsutomu Higashijima, J. Biol. Chem. 1992, 267, 16237-16243); NPY(Y1) (e.g., Novel Analogs of Neuropeptide Y with a Preference for the Y1-receptor, Richard M. Soll, Michaela, C. Dinger, Ingrid Lundell, Dan Larhammer, Annette G. Beck-Sickinger, Eur. J. Biochem. 2001, 268, 2828-2837; 99mTc-Labeled Neuropeptide Y Analogs as Potential Tumor Imaging Agents, Michael Langer, Roberto La Bella, Elisa Garcia-Garayoa, Annette G. Beck-Sickinger, Bioconjugate Chem. 2001, 12, 1028-1034; Novel Peptide Conjugates for Tumor-Specific Chemotherapy, Michael Langer, Felix Kratz, Barbara Rothen-Rutishauser, Heidi Wnderli-Allenspach, Annette G. Beck-Sickinger, J. Med. Chem. 2001, 44, 1341-1348); oxytocin; endothelin A and endothelin B; bradykinin; Endothelial Growth Factor (EGF); Interleukin-1 (Anti-IL-1 Activity of Peptide Fragments of IL-1 Family Proteins, I. Z. Siemion, A. Kluczyk, Zbigtniew Wieczorek, Peptides 1998, 19, 373-382); and cholecystokinin (CCK-B) (Cholecystokinin Receptor Imaging Using an Octapeptide DTPA-CCK Analog in Patients with Medullary Thyroid Carcinoma, Eur. J. Nucl Med. 200, 27, 1312-1317).

Literature which gives a general review of targeting moieties can be found, for example, in the following: The Role of Peptides and Their Receptors as Tumor Markers, Jean-Claude Reubi, Gastrointestinal Hormones in Medicine, p. 899-939; Peptide Radiopharmaceutical in Nuclear Medicine, D. Blok, R. I. J. Feitsma, P. Vermeij, E. J. K. Pauwels, Eur. J. Nucl Med. 1999, 26, 1511-1519; and Radiolabeled Peptides and Other Ligands for Receptors Overexpressed in Tumor Cells for Imaging Neoplasms, John G. McAfee, Ronald D. Neumann, Nuclear Medicine and Biology, 1996, 23, 673-676 (somatostatin, VIP, CCK, GRP, Substance P, Galanan, MSH, LHRH, Arginine-vasopressin, endothelin). All of the aforementioned literature in the preceding paragraphs are herein incorporated by reference in their entirety.

Other targeting moiety references include the following: Co-expressed peptide receptors in breast cancer as a molecular basis of in vivo multireceptor tumor targeting. Jean Claude Reubi, Mathias Gugger, Beatrice Waser. Eur. J. Nucl Med. 2002, 29, 855-862, (includes NPY, GRP); Radiometal-Binding Analogs of Leutenizing Hormone Releasing Hormone PCT/US96/08695 (LHRH); PCT/US97/12084 (WO 98/02192) (LHRH); PCT/EP90/01169 (radiotherapy of peptides); WO 91/01144 (radiotherapy of peptides); and PCT/EP00/01553 (molecules for the treatment and diagnosis of tumors), all of which are herein incorporated by reference in their entirety.

Additionally, analogs of a targeting moiety can be used. These analogs include molecules that target a desired site receptor with avidity that is less than, or more preferably, greater than or equal to, the targeting moiety itself, as well as muteins, retropeptides and retro-inverso-peptides of the targeting moiety. One of ordinary skill will appreciate that these analogs may also contain modifications which include substitutions, and/or deletions and/or additions of one or several amino acids, insofar that these modifications do not significantly negatively alter the biological activity of the moieties described therein. These substitutions may be carried out by replacing one or more amino acids by their synonymous amino acids. Synonymous amino acids within a group are defined as amino acids that have sufficiently similar physicochemical properties to allow substitution between members of a group in order to preserve the biological function of the molecule. Synonymous amino acids as used herein include synthetic derivatives of these amino acids (such as for example the D-forms of amino acids and other synthetic derivatives.

Deletions or insertions of amino acids may also be introduced into the defined sequences provided they do not significantly negatively alter the biological functions of said sequences. Preferentially such insertions or deletions should be limited to 1, 2, 3, 4 or 5 amino acids and should not remove or physically disturb or displace amino acids which are critical to the functional conformation. Muteins of the peptides or polypeptides described herein may have a sequence homologous to the sequence disclosed in the present specification in which amino acid substitutions, deletions, or insertions are present at one or more amino acid positions. Muteins may have a biological activity that is at least 40%, preferably at least 50%, more preferably 60-70%, most preferably 80-90% of the peptides described herein. However, they may also have a biological activity greater than the peptides specifically exemplified, and thus do not necessarily have to be identical to the biological function of the exemplified peptides. Analogs of targeting moieties also include peptidomimetics or pseudopeptides incorporating changes to the amide bonds of the peptide backbone, including thioamides, methylene amines, and E-olefins. Also peptides or other chemical compounds based on the structure of a targeting moiety or its peptide analogs with amino acids replaced by N-substituted hydrazine carbonyl compounds (also known as aza amino acids) are included in the term analogs as used herein.

As examples, the targeting moiety may be attached to the linker via the N or C terminus or via attachment to the epsilon nitrogen of lysine, the gamma nitrogen or ornithine or the second carboxyl group of aspartic or glutamic acid.

In an exemplary embodiment, the targeting peptide Q is LHRH or an analog or derivative thereof. For example, it is well known in the art that position 6 of LHRH agonists may be substituted with different functional groups, such as, for example D-Lysine. In a preferred embodiment, the targeting peptide Q is an LHRH analog of the formula PGlu-His-Trp-W-Tyr-DLys-X—Y-Pro-Z, wherein
W=Ser, NMeSer, or Thr.
X=Leu, NMeLeu, t-ButylGly.
Y=Arg, Arg(Et2), Cit, Lys(isopropyl).
Z=Gly-NH₂, NHEthyl, Azagly-NH₂.

Linkers of the invention coupled to glycine and D-Lysine can be attached to the LHRH analog at position 6.

In a preferred embodiment, the W, X, Y or Z components in the above formulas are W=Ser, X=Leu, Y=Arg and Z=Gly-NH₂.

In another embodiment, Q is a peptide that targets a receptor in the GRP receptor family, such as an analog or derivative of GRP or bombesin. Such targeting peptides are discussed in co-pending U.S. Ser. No. 10/341,577 filed Jan. 13, 2003, as well as in U.S. Pat. No. 6,200,546, U.S. 2002/0054855, WO 02/87637, and U.S. 2003/0224998, which are hereby incorporated by reference herein in their entirety. Compounds containing particular linkers and targeting peptides which target the GRP receptor are disclosed in U.S. Ser. No. 10/341,577 and PCT/US03/41328, which applications are hereby incorporated by reference herein in their entirety. These compounds may demonstrate unexpectedly superior pharmacokinetics and tumor uptake in animal models.

The targeting peptide can be prepared by various methods depending upon the selected chelator and linker. The peptide can generally be most conveniently prepared by techniques generally established and known in the art of peptide synthesis, such as the solid-phase peptide synthesis (SPPS) approach. Solid-phase peptide synthesis (SPPS) involves the stepwise addition of amino acid residues to a growing peptide chain that is linked to a solid support or matrix, such as polystyrene. The C-terminal residue of the peptide is first anchored to a commercially available support with its amino group protected with an N-protecting agent such as a t-butyloxycarbonyl group (Boc) or a fluorenylmethoxycarbonyl (Fmoc) group. The amino protecting group is removed with suitable deprotecting agents such as TFA in the case of Boc or piperidine for Fmoc and the next amino acid residue (in N-protected form) is added with a coupling agent such as N,N′-dicyclohexylcarbodiimide (DCC), or N,N′-diisopropylcarbodiimide or 2-(1H-benzotriazol-1-yl)-1,1,3,3-tetramethyluronium hexafluorophosphate (HBTU). Upon formation of a peptide bond, the reagents are washed from the support. After addition of the final residue, the peptide is cleaved from the support with a suitable reagent such as trifluoroacetic acid (TFA) or hydrogen fluoride (HF).

The linker may then be coupled to form a conjugate by reacting the free amino group of a selected residue of the targeting moiety with an appropriate functional group of the linker, chelating compound or complex. The entire construct of linker and targeting moiety discussed above may also be assembled on resin and then cleaved by agency of suitable reagents such as trifluoroacetic acid or HF, as well.

Regardless of the targeting moiety used, it may be attached to the chelating compound, linker or complex at the most advantageous time. For example, the targeting moiety can be attached to the 1) chelating compound itself before the metal is added, 2) the complex comprising the chelating compound and the metal, 3) the linker (which product of the linker/targeting moiety is then attached to the chelating compound or the complex), 4) the linker/chelating compound, or 5) the linker/complex, depending on which process provides the most advantages in terms of yield and ease in accomplishing the attachment.

### Labeling and Administration of Compounds

Incorporation of the metal within the compound can be achieved by various methods commonly known in the art of coordination chemistry.

For example, when the metal is a paramagnetic metal, the labeling can be accomplished by the methods described herein in the specific examples. Additional methods are well known to persons of skill in the art.

A conjugate labeled with a paramagnetic metal, such as Gd, can be prepared for administration to a mammal, including human patients or subjects, by intravenous, subcutaneous or intraperitoneal injection in a pharmaceutically acceptable carrier and/or solution such as salt solutions like isotonic saline. The particular dosage necessary to provide a desired image can be determined by a person of skill in the art.

A conjugate labeled with a radionuclide metal can be prepared for administration to a mammal, including human patients or subjects, by intravenous, subcutaneous or intraperitoneal injection in a pharmaceutically acceptable carrier and/or solution such as salt solutions like isotonic saline. Radiolabeled scintigraphic imaging agents provided by the present invention are provided having a suitable amount of radioactivity. In forming diagnostic radioactive complexes, it is generally preferred to form radioactive complexes in solutions containing radioactivity at concentrations of from about 0.01 millicurie (mCi) to 100 mCi per mL. Generally, the unit dose to be administered has a radioactivity of about 0.01 mCi to about 100 mCi, preferably 1 mCi to 30 mCi. The solution to be injected at unit dosage is from about 0.01 mL to about 10 mL. The amount of labeled conjugate appropriate for administration is dependent upon the distribution profile of the chosen conjugate in the sense that a rapidly cleared conjugate may need to be administered in higher doses than one that clears less rapidly. In vivo distribution and localization can be tracked by standard scintigraphic techniques at an appropriate time subsequent to administration; typically between thirty minutes and 180 minutes depending upon the rate of accumulation at the target site with respect to the rate of clearance at non-target tissue. For example, after injection of the diagnostic radionuclide-labeled compounds of the invention into the patient, a gamma camera calibrated for the gamma ray energy of the nuclide incorporated in the imaging agent can be used to image areas of uptake of the agent and quantify the amount of radioactivity present in the site. Imaging of the site in vivo can take place in a few minutes. However, imaging can take place, if desired, hours or even longer, after the radiolabeled compound is injected into a patient. In most instances, a sufficient amount of the administered dose will accumulate in the area to be imaged within about 0.1 hour to permit the taking of scintiphotos.

The compounds of the present invention can be administered to a patient alone or as part of a composition that contains other components such as excipients, diluents, radical scavengers, stabilizers, and carriers, all of which are well-known in the art. The compounds can be administered to patients either intravenously or intraperitoneally. In the case of radiopharmaceuticals, compositions of the invention can require radiation stabilizers to prevent radiolytic damage to the compound or complex prior to injection. Radiation stabilizers are known to those skilled in the art, and may include, for example, para-aminobenzoic acid, ascorbic acid, gentistic acid and the like. Particularly preferred stabilizers and formulations are discussed in copending provisional application U.S. Ser. No. 60/489,850, which is hereby incorporated herein in its entirety.

There are numerous advantages associated with the present invention. The compounds made in accordance with the present invention may form stable, well-defined metal complexes and be conjugatable to targeting moieties, with and without linking groups. Another advantage that may be present is that metal that is no longer needed to be the body because imaging is finished or because it does not reach (e.g., does not bind) the desired targeted site or metabolic function may be preferentially excreted efficiently into the urine with minimal retention of the metal in the kidneys.

### Diagnostic and Therapeutic Uses

When labeled with diagnostically and/or therapeutically useful metals, compounds of the present invention can be used to treat and/or detect diseases, such as cancers, including tumors, by procedures established in the art of diagnostics and radiotherapeutics. (Bushbaum, 1995; Fischman et al., 1993; Schubiger et al., 1996; Lowbertz et al., 1994; Krenning et al., 1994).

The diagnostic application of these compounds can be as general imaging agents for MR, radionuclide (e.g., scintigraphic) imaging, x-ray or CT. They may also be used as a first line diagnostic screen for the presence of targeted cells using scintigraphic imaging, as an agent for targeting selected tissue using hand-held radiation detection instrumentation in the field of radioimmuno guided surgery (RIGS), as a means to obtain dosimetry data prior to administration of the matched pair radiotherapeutic compound, and as a means to assess a targeted receptor population as a function of treatment over time. Other applications are indeed possible and will be known to a person of ordinary skill in the art.

The therapeutic application of these compounds can be defined as an agent that will be used as a first line therapy in the treatment of diseases such as cancer, as combination therapy where these radiolabeled agents could be utilized in conjunction with adjuvant chemotherapy (using, for example, one of the other therapeutic moieties discussed herein), and/or as a matched pair therapeutic agent. The matched pair concept refers to a single unmetallated compound which can serve as both a diagnostic and a therapeutic agent depending on the radiometal that has been selected for binding to the appropriate chelate. If the chelator cannot accommodate the desired metals appropriate substitutions can be made to accommodate the different metal while maintaining the pharmacology such that the behavior of the diagnostic compound in vivo can be used to predict the behavior of the radiotherapeutic compound.

### Radiotherapy

Radioisotope therapy involves the administration of a radiolabeled compound in sufficient quantity to damage or destroy the targeted tissue. After administration of the compound (e.g., by intravenous, subcutaneous, or intraperitoneal injection), the radiolabeled pharmaceutical localizes preferentially at the disease site. Once localized, the radiolabeled compound then damages or destroys the diseased tissue with the energy that is released during the radioactive decay of the isotope that is administered.

The design of a successful radiotherapeutic may involve several factors: 1) selection of an appropriate targeting group to deliver the radioactivity to the disease site; 2) selection of an appropriate radionuclide that releases sufficient energy to damage that disease site, without substantially damaging adjacent normal tissues; and 3) selection of an appropriate combination of the targeting group and the radionuclide without adversely affecting the ability of this conjugate to localize at the disease site. For radiometals, this often involves a chelating group that coordinates tightly to the radionuclide, combined with a linker that couples said chelate to the targeting group, and that affects the overall biodistribution of the compound to maximize uptake in target tissues and minimizes uptake in normal, non-target organs. The present invention may be used to provide radiotherapeutic agents that satisfy all three of the above criteria, through proper selection of a targeting group, radionuclide, metal chelate and linker.

Radiotherapeutic agents may contain a chelated 3⁺ metal ion from the class of elements known as the lanthanides (elements of atomic number 57-71) and their analogs (i.e., M³⁺ metals such as yttrium and indium). Typical radioactive metals in this class include the isotopes 90-Yttrium, 111-Indium, 149-Promethium, 153-Samarium, 166-Dysprosium, 166-Holmium, 175-Ytterbium, and 177-Lutetium.

General methods for coupling the polyaza macrocycle compounds of this invention to targeting groups through a linker (e.g., by activation of one of the carboxylates of the polyaza macrocycle to form an active ester, which is then reacted with an amino group on the linker to form a stable amide bond), are known to those skilled in the art (see e.g., Tweedle et al. U.S. Pat. No. 4,885,363).

The selection of a proper nuclide for use in a particular radiotherapeutic application depends on many factors, including:

a. Physical half-life—This should be long enough to allow synthesis and purification of the radiotherapeutic construct from radiometal and conjugate, and delivery of said construct to the site of injection, without significant radioactive decay prior to injection. Preferably, the radionuclide should have a physical half-life between about 0.5 and 8 days.

b. Energy of the emission(s) from the radionuclide—Radionuclides that are particle emitters (such as alpha emitters, beta emitters and Auger electron emitters) are particularly useful, as they emit highly energetic particles that deposit their energy over short distances, thereby producing highly localized damage. Beta emitting radionuclides are particularly preferred, as the energy from beta particle emissions from these isotopes is deposited within 5 to about 150 cell diameters. Radiotherapeutic agents prepared from these nuclides are capable of killing diseased cells that are relatively close to their site of localization, but cannot travel long distances to damage adjacent normal tissue such as bone marrow.

c. Specific activity (i.e., radioactivity per mass of the radionuclide)—Radionuclides that have high specific activity (e.g., generator produced 90-Y, 111-In, 177-Lu) are particularly preferred. The specific activity of a radionuclide is determined by its method of production, the particular target that is used to produce it, and the properties of the isotope in question.

Many of the lanthanides and lanthanoids include radioisotopes that have nuclear properties that make them suitable for use as radiotherapeutic agents, as they emit beta particles. Some of these are listed in the table below.

| | | | | Approximate | |
|---|---|---|---|---|---|
| | | | | range of b- | |
| | | | | particle | |
| | Half-Life | Max b-energy | Gamma energy | (cell | |
| Isotope | (days) | (MeV) | (keV) | diameters) | |
| ¹⁴⁹Pm | 2.21 | 1.1 | 286 | 60 | |
| ¹⁵³Sm | 1.93 | 0.69 | 103 | 30 | |
| ¹⁶⁶Dy | 3.40 | 0.40 | 82.5 | 15 | |
| ¹⁶⁶Ho | 1.12 | 1.8 | 80.6 | 117 | |
| ¹⁷⁵Yb | 4.19 | 0.47 | 396 | 17 | |
| ¹⁷⁷Lu | 6.71 | 0.50 | 208 | 20 | |
| ⁹⁰Y | 2.67 | 2.28 | — | 150 | |
| ¹¹¹In | 2.810 | Auger electron | 173,247 | <5 | μm |
| | | emitter | | | |

Pm: Promethium,
Sm: Samarium,
Dy: Dysprosium,
Ho: Holmium,
Yb: Ytterbium,
Lu: Lutetium,
Y: Yttrium,
In: Indium

Methods for the preparation of radiometals such as beta-emitting lanthanide radioisotopes are known to those skilled in the art, and have been described elsewhere (e.g., Cutler C S, Smith C J, Ehrhardt G J.; Tyler T T, Jurisson S S, Deutsch E. “Current and potential therapeutic uses of lanthanide radioisotopes.” Cancer Biother. Radiopharm. 2000; 15(6): 531-545). Many of these isotopes can be produced in high yield for relatively low cost, and many (e.g., ⁹⁰Y, ¹⁴⁹Pm, ¹⁷⁷Lu) can be produced at close to carrier-free specific activities (i.e., the vast majority of atoms are radioactive). Since non-radioactive atoms can compete with their radioactive analogs for binding to receptors on the target tissue, the use of high specific activity radioisotope is important, to allow delivery of as high a dose of radioactivity to the target tissue as possible.

### Dosages and Additives

Proper dose schedules for the compounds of the present invention are known to those skilled in the art. The compounds can be administered using many methods which include, but are not limited to, a single or multiple IV or IP injections.

For example, the complexes made with the compounds of formula (I), (II), (III) and (IV) can be administered as MRI contrast agents parenterally, preferably formulated as a sterile aqueous solution or suspension, whose pH range from 6.0 to 8.0. Said aqueous solutions or suspensions can be administered in concentration ranging from 0.002 to 1.0 molar. These formulations can be freeze dried and supplied as they are to be reconstituted before use.

The complexes made with the compounds of formula (I), (II), (III) and (IV) also can be administered as radionuclide (e.g., scintigraphic) imaging, x-ray and CT contrast agents parenterally, preferably formulated as a sterile aqueous solution or suspension, whose pH range from 6.0 to 8.0. Said aqueous solutions or suspensions can be administered in concentration ranging from 0.002 to 1.0 M. These formulations can be freeze dried and supplied as they are to be reconstituted before use, before or after adding a radionuclide.

The complexes made with the compounds of formula (I), (II), (III) and (IV) also can be administered as radiopharmaceuticals parenterally, preferably formulated as a sterile aqueous solution or suspension, whose pH range from 6.0 to 8.0. Said aqueous solutions or suspensions can be administered in concentration ranging from 0.002 to 1.0 M. These formulations can be freeze dried and supplied as they are to be reconstituted before use, before or after adding a radionuclide.

In addition, for radiopharmaceutical applications, one would use a quantity of radioactivity that is sufficient to permit imaging, or in the case of radiotherapy, to cause damage or ablation of the targeted tissue, but not so much that substantive damage is caused to non-target (normal tissue). The quantity and dose required for scintigraphic imaging is discussed above. The quantity and dose required for radiotherapy is also different for different constructs, depending on the energy and half-life of the isotope used, the degree of uptake and clearance of the agent from the body and the mass of the tumor. In general, doses can range from a single dose of about 30-50 mCi to a cumulative dose of up to about 3 Curies.

The MRI agent and radiopharmaceutical compositions of the invention can include physiologically acceptable buffers and other excipients. In addition, radiopharmaceutical compositions can include or require radiation stabilizers to prevent radiolytic damage to the compound prior to injection. Radiation stabilizers are known to those skilled in the art, and may include, for example, para-aminobenzoic acid, ascorbic acid, gentistic acid and the like. Particularly preferred stabilizers are disclosed in co-pending U.S. Ser. No. 60/489,850, which is hereby incorporated by reference herein in its entirety.

A single or multi-vial kit that contains all of the components needed to prepare the MRI agents and radiopharmaceuticals of this invention is an integral part of this invention.

A single-vial kit for a radiopharmaceutical preferably contains a chelating compound/optional linker/optional targeting peptide conjugate, a source of stannous salt (if reduction is required), or other pharmaceutically acceptable reducing agent, and is appropriately buffered with pharmaceutically acceptable acid or base to adjust the pH to a value of about 3 to about 9. The quantity and type of reducing agent used will depend highly on the nature of the exchange complex to be formed. The proper conditions are well known to those that are skilled in the art. It is preferred that the kit contents be in lyophilized form. Such a single vial kit may optionally contain labile or exchange ligands such as glucoheptonate, gluconate, mannitol, malate, citric or tartaric acid and can also contain reaction modifiers such as diethylenetriamine-pentaacetic acid (DPTA), ethylenediamine tetraacetic acid (EDTA), or α, β, or γ-cyclodextrin that serve to improve the radiochemical purity and stability of the final product. The kit may also contain stabilizers, bulking agents such as mannitol, that are designed to aid in the freeze-drying process, and other additives known to those skilled in the art. Particularly preferred stabilizers are disclosed in co-pending U.S. Ser. No. 60/489,850, which is hereby incorporated by reference herein in its entirety.

A multi-vial kit preferably contains the same general components but employs more than one vial in reconstituting the radiopharmaceutical. For example, one vial may contain all of the ingredients that are required to form a labile metal complex on addition of pertechnetate (e.g., the stannous source or other reducing agent). Pertechnetate is added to this vial, and after waiting an appropriate period of time, the contents of this vial are added to a second vial that contains the chelator and targeting peptide, as well as buffers appropriate to adjust the pH to its optimal value. After a reaction time of about 5 to 60 minutes, the complexes of the present invention are formed. It is advantageous that the contents of both vials of this multi-vial kit be lyophilized. As above, reaction modifiers, exchange ligands, stabilizers, bulking agents, etc., may be present in either or both vials.

### General Preparation of Compounds

The compounds of the present invention can be prepared by various methods depending upon the selected chelator. Novel methods are described below in the examples.

Any peptide portions used as linkers or targeting moieties can be most conveniently prepared by techniques generally established and known in the art of peptide synthesis, such as the solid-phase peptide synthesis (SPPS) approach. Because it is amenable to solid phase synthesis, employing alternating Fmoc protection and deprotection is the preferred method of making short peptides. Recombinant DNA technology is preferred for producing proteins and long fragments thereof.

Solid-phase peptide synthesis (SPPS) involves the stepwise addition of amino acid residues to a growing peptide chain that is linked to a solid support or matrix, such as polystyrene. The C-terminal residue of the peptide is first anchored to a commercially available support with its amino group protected with an N-protecting agent such as a t-butyloxycarbonyl group (Boc) or a fluorenylmethoxycarbonyl (Fmoc) group. The amino protecting group is removed with suitable deprotecting agents such as TFA in the case of Boc or piperidine for Fmoc and the next amino acid residue (in N-protected form) is added with a coupling agent such as N,N′-dicyclohexylcarbodiimide (DCC). Upon formation of a peptide bond, the reagents are washed from the support. After addition of the final residue, the peptide is cleaved from the support with a suitable reagent such as trifluoroacetic acid (TFA) or hydrogen fluoride (HF).

The following preferred embodiments and Examples are provided to give illustrations of the present invention and are not meant to be limiting.

### EXAMPLES

### Examples 1-6 below are preferred compounds of this invention that are capable of chelating metals

### Example 1

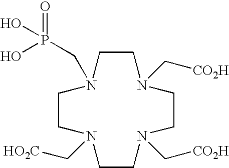 [0107] 10-Phosphonomethyl-1,4,7,10-tetraazacyclododecane-1,4,7-triacetic acid (MPDO3A)

This compound exhibits some of the advantages of this invention. Replacement of one of the methyl arms of DOTA with a phosphonomethyl arm via the teaching described herein provides a polyaza macrocycle, MPDO3A, which exhibits a surprisingly high relaxivity when it complexes gadolinium when compared to DOTA or DO3A.

| | | r₁mM⁻¹.s⁻¹ |
|---|---|---|
| Compound | r₁mM⁻¹.s⁻¹ | in Sucrose 80 |
| DOTA | 3.5 | 35 |
| MPDO3A (Na) (Example 1) | 4.1 | 65 |
| EPDO3A (Na) (Example 2) | 4.1 | 64 |

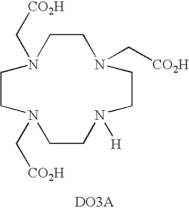

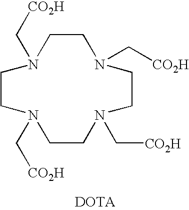

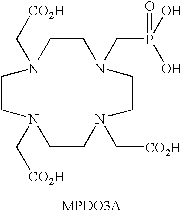

The structure of MPDO3A in solution when it is complexed with gadolinium includes the following:

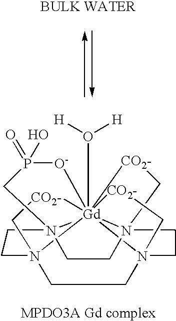

### Example 2

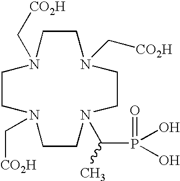

10-(1-phosphonoethyl)-1,4,7,10-tetrazacycododecane-1,4,7-triacetic acid. See the table immediately above for relaxivity data for this compound (EPDO3A) when it is complexed with gadolinium.

### Example 3

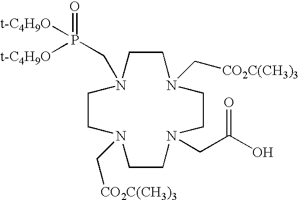 [0113] 10-[[Bis(1,1-dimethylethoxy)phosphinyl]methyl]-1,4,7,10-tetraazacyclododecane-1,4,7-triacetic acid 1,7-bis(1,1-dimethylethyl)ester

### Example 4

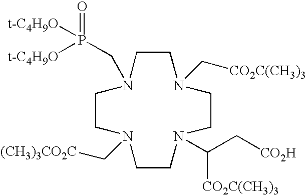 [0115] 10-[[Bis(1,1-dimethylethoxy)phosphinyl]methyl]-α′-(carboxymethyl)-1,4,7,10-tetraazacyclododecane-1,4,7-triacetic acid α,α′,α″-tris(1,1-dimethylethyl)ester

### Example 5

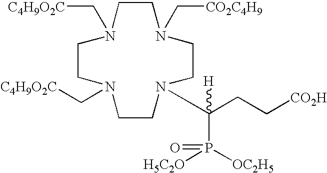 [0117] 10-[[1-[Bis(1,1-dimethylethoxy)phosphinyl]-3-carboxyl]propyl]-1,4,7,10-tetraazacyclododecane-1,4,7-triacetic α,α′,α″-tris(1,1-dimethylethyl)ester

### Example 6

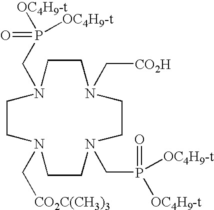 [0119] 4,10-Bis[[bis(1,1-dimethylethoxy)phosphinyl]methyl]-1,4,7,10-tetraazacyclododecane-1,7-diacetic (1,1-dimethylethyl)ester

### Example 7

### Synthesis of Compound of Example 1 (MPDO3A)

Scheme 1 shows a synthetic route used to make MPDO3A (10-Phosphonomethyl-1,4,7,10-tetraazacyclododecane-1,4,7-triacetic acid, gadolinium salt (3).

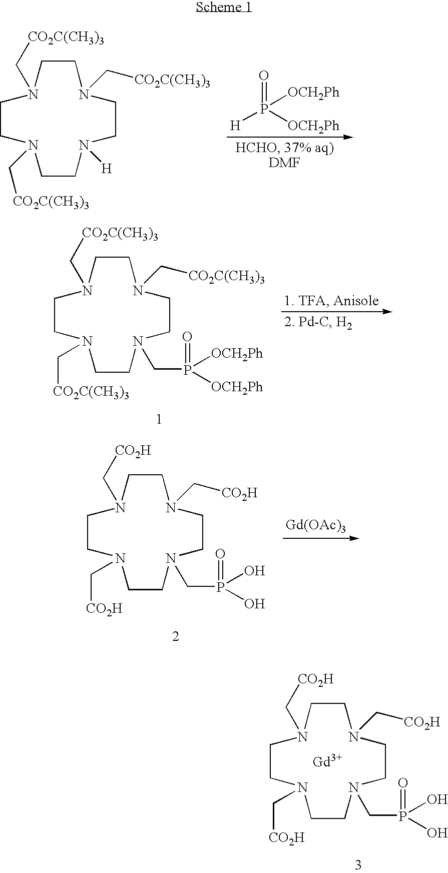

Treatment of DO3A-tris t-butyl ester with formaldehyde and dibenzyl phosphite in DMF at room temperature furnished the adduct 1 in 60% yield. Deprotection followed by anion exchange chromatographic purification afforded MPDO3A 3 in 40% yield.

### Example 7A

This example shows the synthetic route used to make 10-[[bis(phenylmethoxy)phosphinyl]methyl]-1,4,7,10-tetraazacyclododecane-1,4,7-triacetic acid tris(1,-dimethylethyl)ester (1) in Scheme 1 above.

To a solution of a free base of 1,4,7,10-tetraazacyclododecane-1,4,7-triacetic-tris-(1,1-dimethylethyl)ester (100 mg, 0.19 mmol) in DMF (2 ml) was added dibenzylphosphite (Aldrich, 0.050 ml, 0.24 mmol). The solution was heated to 100° C. and 37% of aqueous formaldehyde (0.16 ml, 2.1 mmol) was added drop-wise. The resulting mixture was stirred at 100° C. for 4 h. DMF was removed by vacuum. Purification of the residue by silica chromatography using MeOH/CHCl₃/NH₄OH (5:95:2) afforded the pure product 1 (70 mg, yield 45%).

TLC: silica gel, R_{f} 0.65, MeOH: CHCl₃: NH₄OH 10:90:3, visualized by I₂.

¹HNMR (CDCl₃, δ): 1.45 (s, 18H); 1.49 (s, 9H); 2.50-3.75 (m, 24H); 5.00 (2 s,); 7.20-7.41 (m, 10H).

MS: (M−Bn+H)⁺ at 699.3.

### Example 7B

This example shows the synthetic route used to make 10-phosphonomethyl-1,4,7,10-tetraazacyclododecane-1,4,7-triacetic acid, (2) in Scheme 1 above.

To a solution of 1 (1.4 g, 1.8 mmol) in TFA (Aldrich, 68 ml) was added anisole (Aldrich, 12 ml). The solution was stirred at room temperature for 16 h. TFA was removed by vacuum. Anisole was removed by azeotropic evaporation with water several times. The oil was then dissolved in MeOH/H₂O (62/15 mL), and directly hydrogenated at 50 psi for 16 h, using Pd/C (10%, wet, 390 mg). The mixture was filtered through a celite cake and the cake was washed thoroughly by MeOH and H₂O. Evaporation of solvents afforded the crude material. The crude product in water (pH adjusted to 7) was then applied onto a 70 ml AG1-X2 ion exchange resin (formate form) column. The resin column was eluted with H₂O, then with formic acid (0.05-1 M). The desired material eluted at about 500 mM of formic acid. The fractions containing the product were combined and evaporation of solvents afforded pure compound 2 as a white solid (600 mg, yield 76% for two steps).

HPLC1: Column: PRP-X100. Conditions: 3% CH₃CN/50 mM NaH₂PO₄ (pH6.5); UV at 220 nm; flow rate 1 ml/min. t_{R}: 12.63 min.

HPLC2: Column: PRP-X100. Conditions: Cu method; 3% CH₃CN/50 mM NaH₂PO₄ (pH6.5); UV at 290 nm; flow rate ml/min. t_{R}: 12.28 min.

MS: (M+H)⁺ at 441.2.

Elemental analysis: Found: C, 38.99; H, 6.62; N, 11.78. Calc'd for C₁₅H₂₉N₄O₉P.1.35H₂O: C, 38.77; H, 6.88; N, 12.06.

HNMR (D₂O, δ): 3.12-3.80 (m, NCH₂'s).

### Example 7C

This example shows the synthetic route used to make the gadolinium complex of 10-phosphonomethyl-1,4,7,10-tetraazacyclododecane-1,4,7-triacetic acid, (3) in Scheme 1 above.

The ligand 2 (50 mg, 0.114 mmol) was dissolved in H₂O (3 ml) and the pH of the solution was adjusted to 6.5 by adding 1 N NaOH. To this solution was added a solution of Gd (OAc)₃ (Aldrich, 50.8 mg, 0.125 mmol) in H₂O (3 ml). The pH of the solution was maintained at 6.5 and it was stirred at 40° C. for 4 h and room temperature overnight. The reaction was monitored by HPLC. Water was evaporated and the crude material was applied onto a 30 ml of DEAD-sephadex A-25. It was eluted with 5-1200 mM NH₄HCO₃. The compound eluted out at 250 mM of NH₄HCO₃. The fractions were combined and solvent evaporated by vacuum. 79 mg of 3 was obtained as an off white solid (yield 85.1%).

HPLC1: Column: Nucleosil C-18. Conditions: 3% CH₃CN/Tris-EDTA (pH7); fluorescence Ex/Em 280/320 nm; flow rate 1 ml/min. t_{R}: 3.83 min.

HPLC2: Column: PRP-X100. Conditions: 3% CH₃CN/50 mM NaH₂PO₄ (pH6.5); UV at 220 nm; flow rate 1 ml/min. t_{R}: 2.56 min.

MS: (M+H)⁺ at 596.2.

Elemental Analysis Found: C, 35.94; H, 6.64; N, 9.56. Calc'd for C₁₅H₂₆N₄O₉PGd.1.58Et₃N.3.3H₂O: C, 36.12; H, 6.97; N, 9.60.

### Example 8

### Synthesis of Compound of Example 2 (10-(1-phosphonoethyl)-1,4,7,10-tetrazacycododecane-1,4,7-triacetic acid)

Scheme 2 shows the synthetic route used to make the compound of Example 2 (10-(1-phosphonoethyl)-1,4,7,10-tetrazacycododecane-1,4,7-triacetic acid) above.

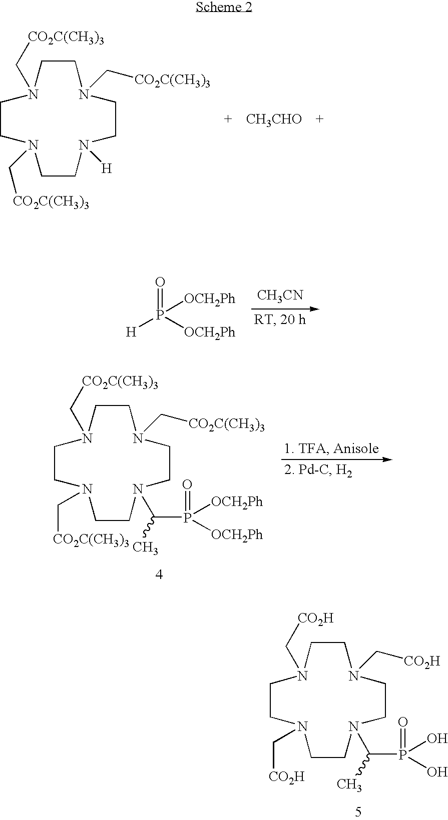

Treatment of 1,4,7,10-tetraazacyclododecane-1,4,7-triacetic-tris-(1,1-dimethylethyl)ester with acetaldehyde and dibenzyl phosphite in acetonitrile at room temperature furnished the protected ligand 4 in 60% yield. Deprotection followed by anion exchange chromatographic purification afforded 5 in 44% yield.

### Example 8A

This example shows the synthetic route used to make 10-[1-[bis(phenylmethoxy)phosphinyl]ethyl]-1,4,7,10-tetraazacyclododecane-1,4,7-triacetic acid tris(1,1-dimethylethyl)ester, (4) in Scheme 2 above.

To a solution of a free base of 1,4,7,10-tetraazacyclododecane-1,4,7,-triacetic-tris-(1,1-dimethyl)ester (2.6 5 g, 5 mmol) in acetonitrile (10 mL) dibenzylphosphite (1.4 g, 5.5 mmol) was added. Acetaldehyde (308 mg, 7 mmol) was then added quickly. Heat is evolved and the solution turned yellowish. It was stirred at room temperature overnight. The reaction mixture was diluted with acetonitrile and dried (MgSO₄). Solvent was removed by vacuum. Purification by silica gel chromatography using MeOH, CHCl₃) afforded the pure product 4 (2.4 g, Yield 60%).

TLC: Silica gel, Fro, 0.3, MeOH: CHCl₃ (1:10), Visualized by I₂.

1H NMR: (CDCl3, δ) 1.19-1.31 (m, 3H,); 1.45 (3 s, 27H); 2.60-3.41 (m, 23H); 4.70-5.10 m, 4H); 7.35 (2 S, 10H).

MS: (M+H)=803; M+Na)=825.

### Example 8B

This example shows the synthetic route used to make 10-(1-phosphonoethyl)-1,4,7,10-tetrazacycododecane-1,4,7-triacetic acid, (5) in Scheme 2 above.

To a solution of 4 (1.94 g, 2.4 mmol) in TFA (90 mL) was added anisole (12 mL). The solution was stirred at room temperature for 16 h. TFA was removed by vacuum. Anisole was removed by azeotropic distillation with water. The brown oil was dissolved in MeOH/water (4:1, 100 mL) and directly hydrogenated at 50 psi for 16 h using Pd—C 10%. 510 mg. The mixture was then filtered through a celite cake and the cake was washed thoroughly by methanol and water. Evaporation of solvents afforded the crude material. The crude product in water (pH adjusted to 7) was then applied on to AG1-X2 ion exchange resin (formate form) column. The resin column was eluted with water, then with formic acid (0.05-1.75 mM). The desired material eluted at 450 M of formic acid. The fractions containing the desired product were combined and evaporation of solvents afforded pure compound 5 as a white solid. (470 mg). Yield 44.3%.

HPLC: Column PRP-X 100. Conditions: 3 CH3CN in 50 mM NaH2PO4 (pH 7); UV at 220 nm; Flow rate 1 mL/min Tr: 8.33 min.

MS: 455 (M+H)+; 453 (M−H).

Elemental analysis: Calc'd for C16H31N4PO9 1.3H2O, C, 40.09; H, 7.10; N, 11.69; P, 6.46. Found: C, 39.85; H, 6.66; N, 11.79; P, 6.30.

IR (KBr, cm-1): 3432 (OH; 1719, 1634 (C═O).

¹H NMR (D2O) δ: 1.22 (br, 3H, 3.05-3.80 (m, 23 h).

¹³CNMR (D2O) d: 1 1.5 (br) 48.0-572 (br); 172-175.0 (br).

³¹PNMR (d20) d, relative to H3PO4): 11.32 (s)

### Example 9

### Synthesis of the Compound of Example 3 (10-[[Bis(1,1-dimethylethoxy)phosphinyl]methyl]-1,4,7,10-tetraazacyclododecane-1,4,7-triacetic acid 1,7-bis(1,1-dimethylethyl)ester)

Scheme 3 shows the synthetic route used to make the compound of Example 3 above (10-[[Bis(1,1-dimethylethoxy)phosphinyl]methyl]-1,4,7,10-tetraazacyclododecane-1,4,7-triacetic acid 1,7-bis(1,1-dimethylethyl)ester). Scheme 3 can be used in the synthesis of DO3A analogs bearing a carboxylic acid group along with phosphonic acid protection that may aid conjugation to a linker or targeting moiety, or other diagnostic or therapeutic moiety.

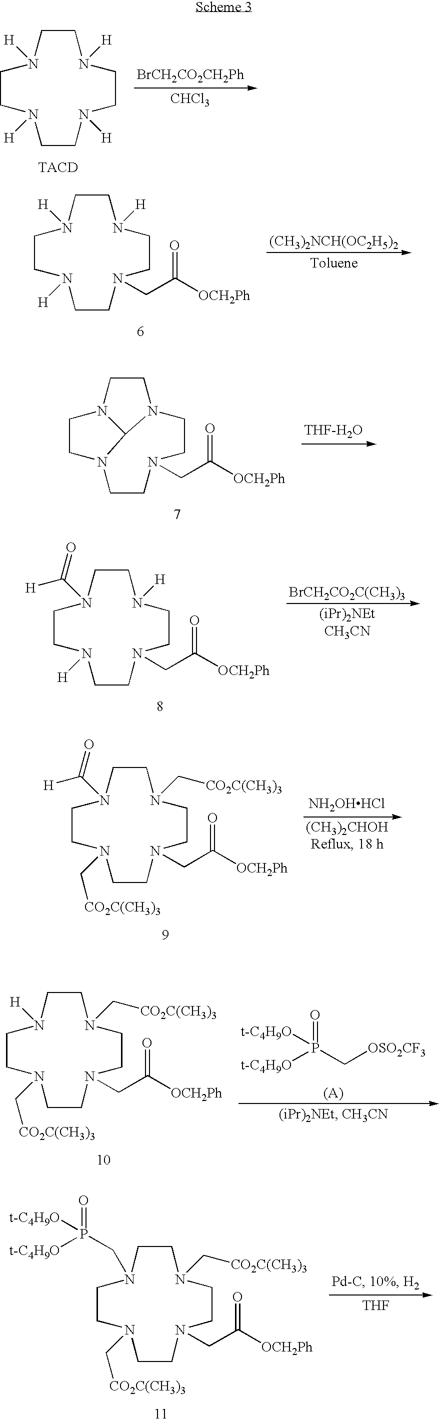

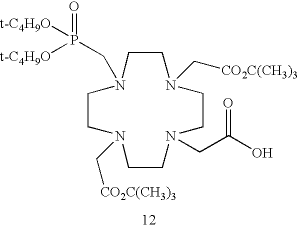

Monoalkylation of the TACD (2.5 equivalents) with benzyl 2-bromoacetate afforded the mono benzyl ester which was purified by column chromatography using methylene chloride methanol and ammonia. The mono benzyl ester 6 was obtained in 30% yield.

Compound 6 was then reacted with N,N-dimethylformamide diethyl acetal in toluene to give the tricycle 7 in near quantitative yield. The tricycle 7 was treated with water-THF mixture for 24 h to give the formyl derivative 8. Alkylation of the crude formyl compound with tert-butyl bromoacetate followed by purification of the alkylated product by column chromatography gave the fully protected macrocycle 9 in 50% yield.

Deformylation using hydroxylamine hydrochloride in isopropanol afforded the ditertiary-butyl ester 10 in 78% yield.

Di-tert-butyl hydroxymethylphosphonate 13 required to complete the synthesis of 7-MPDO3A was prepared by reacting tertiary butyl phosphite with aqueous formaldehyde in the presence of triethylamine. Di-tert-butyl hydroxymethylphosphonate 13 was then converted to the triflate 14 using trifluoromethanesulfonyl chloride and sodium hydride in ether at −78° C.

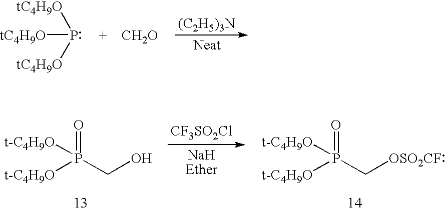

Alkylation of ditertiary-butyl ester 10 with triflate 14 in acetonitrile in the presence of diisopropylethylamine gave the benzyl ester 11 in 56% yield.

Debenzylation of 11 using Pd—C 10%, in THF afforded the conjugacatable ligand 12.

### Example 9A

This example shows the synthetic route used to make 1,4,7,10-tetraazacyclododecane-1-acetic acid phenylmethyl ester, (6) in Scheme 3 above.

To a solution of TACD (21.5 g, 0.125 mol) in CHCl₃ (200 mL) was added benzyl-2-bromoacetate (11.45 g, 7.92 mL, 0.05 mol) in CHCl₃ (50 mL) dropwise over a period of 2 h and the reaction mixture was allowed to stand at 4° C. for 24 h. Chloroform was removed on a rotary evaporator and the residue was treated with a saturated solution of sodium bicarbonate (200 mL). The mixture was extracted with methylene chloride and the methylene chloride solution was washed with water and the organic layer was removed and dried with sodium sulfate. Methylene chloride was removed to give 6 as a viscous oil, which was dried, under vacuum for 24 h. Yield 12.0 g. (75%). The crude 1,4,7,10-tetraazacyclododecane-1-acetic acid phenylmethyl ester 6 was used in the next step without further purification.

MS: (M+H)⁺=321

¹H-NMR (CDCl₃): δ 2.55 and 2.75 (m, 16H), 3.48 (s, 2H), 5.12 (s, 2H), 7.32 (m, 5H).

### Example 9B

This example shows the synthetic route used to make octahydro-7H,9bH-2a,4a,7,9a-tetraazacycloocta[cd]pentalene-7-acetic acid phenylmethyl ester (7) in Scheme 3 above.

To a solution of the benzyl ester 6 (11.0 g, 0.034 mol) in toluene (300 mL) was added dimethylformamide diethyl acetal (7.3 g, 8.5 mL, 0.049 mol) and the mixture was heated at 85° C. for 12 h. Toluene was evaporated on a rotary evaporator. The yellow oil obtained was treated with dry ether (200 mL). A gummy solid was formed during the addition of ether and the yellow ether solution was filtered through celite. The filter cake was washed with ether (2×100 mL). The combined ether solution was concentrated to give a thick yellow oil. Yield 10.2 g (89%). The crude octahydro-7H,9bH-2a,4a,7,9a-tetraazacycloocta[cd]pentalene-7-acetic acid phenylmethyl ester (7) obtained was used in the next step without further purification.

MS: (M+H)⁺=331

¹H-NMR (CDCl₃): δ 2.60 and 3.09 (m, 16H), 3.52 (s, 2H), 4.81 (s, 1H), 5.12 (s, 2H), 7.32 (m, 5H).

### Example 9C

This example shows the synthetic route used to make 7-formyl-1,4,7,10-tetraazacyclododecane-1-acetic acid phenylmethyl ester, (8) in Scheme 3 above.

To a solution of octahydro-7H,9bH-2a,4a,7,9a-tetraazacycloocta[cd]pentalene-7-acetic acid phenylmethyl ester (7) (10.0 g, 0.06 mol) in THF (100 mL) was added water (25 mL) and the mixture was stirred at room temperature for 18 h. THF-water was removed under vacuum and the light yellow oil of 8 obtained was dried under vacuum. This was used in the next step with out further purification. Yield 9.0 g (85.7%).

MS: (M+H)⁺=349.2

¹H-NMR (CDCl₃): δ 2.60-2.9 and 3.4-3.9 (m and s, 18H), 5.12 (s, 2H), 7.32 (m, 5H), 8.12 (s, 1H).

### Example 9D

This example shows the synthetic route used to make 10-formyl-1,4,7,10-tetraazacyclododecane-1,4,7-triacetic acid 1,7-bis(1,1-dimethylethyl) 4-(phenylmethyl)ester, (9) in Scheme 3 above.

To a solution of 7-formyl-1,4,7,10-tetraazacyclododecane-1-acetic acid phenylmethyl ester (8) (9.0 g, 0.0258 mol) in acetonitrile (80 mL) were added diisopropylethylamine (13.0 g, 17.6 mL, 0.1 mol) and ter-butyl bromoacetate (11.6 g, 8.8 mL, 0.06 mol) and the mixture was stirred at room temperature for 24 h. Acetonitrile was removed on a rotary evaporator and the residue was treated with water (150 mL) and extracted with ethyl acetate (250 mL). The ethyl acetate layer was washed with NaHCO₃ solution (200 mL), water (200 mL) and dried (Na₂SO₄). The ethyl acetate was removed and the oil obtained was chromatographed over silica gel. (The column was packed with 7:3, hexane-ethyl acetate, and eluted with 7:3 hexane-ethyl acetate 600 mL, 50:50 hexane-ethyl acetate 600 mL, and finally with pure ethyl acetate). Fractions containing the product were collected and evaporated to give 9 as a thick yellow oil, which was dried, under vacuum.

Yield 8.7 g (59%).

MS: (M+H)⁺=577.5.

HRMS (FAB) m/z: Calc'd for C₃₀H₄₈N₄O₇ (M+Na⁺): 599.3421. Found: 599.3422.

¹H-NMR (CDCl₃): δ 1.45 (s, 18H), 2.65-2.95 (m, 12H), 3.42 and 3.49 (m, 2H), 5.12 (s, 2H), 7.32 (m, 5H), 8.02 (s, 1H).

### Example 9E

This example shows the synthetic route used to make 1,4,7,10-tetraazacyclododecane-1,4,7-triacetic acid 1,7-bis(1,1-dimethylethyl)4-(phenylmethyl)ester, (10) in Scheme 3 above.

A mixture of 10-formyl-1,4,7,10-tetraazacyclododecane-1,4,7-triacetic acid 1,7-bis(1,1-dimethylethyl)4-(phenylmethyl)ester 9 (5.0 g; 8.6 mmol) and hydroxylamine hydrochloride (0.8 g; 11.5 mmol) and water (0.3 mL) in isopropanol (35 mL) was refluxed for 12 h. The progress of the reaction was monitored by TLC using methylene chloride methanol (95:5). Isopropanol and water were removed on a rotary evaporator and the viscous oil obtained was dried under vacuum. This was treated with chloroform (200 mL) and the light precipitated formed was filtered and the chloroform solution was concentrated to a viscous oil. The oil was dried under vacuum to give a foamy solid. This was titrated with ether (75 mL) and the precipitated white solid was filtered and dried to obtain 10. Yield 3.52 g (74%).

MS: (M+H)⁺=549

¹H-NMR (CDCl₃): [symbol] 1.49 (s, 18H), 2.60-2.9 (m and 16H), 3.45 (s, 4H), 3.52 (s, 2H), 5.12 (s, 2H), 7.32 (m, 5H).

### Example 9F

This example shows the synthetic route used to make 10-[[bis(1,1-dimethylethoxy)phosphinyl]methyl]-1,4,7,10-tetraazacyclododecane-1,4,7-triacetic acid 1,7-bis(1,1-dimethylethyl)4-(phenylmethyl)ester, (11) in Scheme 3 above.

To a mixture of the benzyl ester (10) 3.0 g (5.47 mmol) and diisopropylethylamine 3.25 g (4.38 mL, 25 mmol) in acetonitrile (10 mL) was added the triflate (14) (Phillion, D. P., U.S. Pat. No. 4,740,608 (1988)) 3.0 g (8.42 mmol) in acetonitrile (5 mL), and the mixture was stirred at 40° C. for 6 h. After the reaction, acetonitrile was removed and the residue was treated with a saturated solution of sodium carbonate (25 mL). The mixture was extracted with ethyl acetate (2×100 mL), washed with water (100 mL) and dried (Na₂SO₄). The ethyl acetate was removed and the oil obtained was dried under vacuum. The foamy solid obtained was triturated with hexane (3×75 mL) at 50° C. to give 10-[[bis(1,1-dimethylethoxy)phosphinyl]methyl]-1,4,7,10-tetraazacyclododecane-1,4,7-triacetic acid 1,7-bis(1,1-dimethylethyl)4-(phenylmethyl)ester 11 as a white solid. Yield. 3.0 g (73%). An analytical sample was obtained by crystallizing from hexane ethyl acetate.

MS: (M+H)⁺=755.5.

HRMS (FAB) m/z: Calc'd for C₃₈H₆₇N₄O₉P Na (M+Na): 777.4543. Found: 777.4599.

¹H-NMR (CDCl₃): δ 1.42 (s, 18H), 1.51 (s, 18H), 2.10-2.95 (m, 24H), 5.10 (s, 2H), 7.35 (m, 5H).

### Example 9G

This example shows the synthetic route used to make 10-[[bis(1,1-dimethylethoxy)phosphinyl]methyl]-1,4,7,10-tetraazacyclododecane-1,4,7-triacetic acid 1,7-bis(1,1-dimethylethyl)ester, (12) in Scheme 3 above.

To a solution of 10-[[bis(1,1-dimethylethoxy)phosphinyl]methyl]-1,4,7,10-tetraazacyclododecane-1,4,7-triacetic acid 1,7-bis(1,1-dimethylethyl)4-(phenylmethyl)ester 11 (1.0 g; 7.8 mmol) in THF (20 mL) was added Pd—C 10% (0.5 g, Degussa type˜50% water) and the mixture was hydrogenated at 45 psi for 12 h. The catalyst was filtered through celite and the filter cake was washed with THF (2×30 mL). The combined THF solution was concentrated on a rotary evaporator to give the acid as a thick viscous oil. This was dried under vacuum for 24 h to give a foamy solid and used as such without further purification. Yield. 0.84 g (95%).

MS (FAB): (M+Na)⁺=687. m-Nitrobenzylester (M+H)⁺=801, (M−C₄H₈+H)⁺=743, (M−C₄H₈O+H)⁺=729. (m-Nitrobenzylalcohol was used for the matrix).

HRMS (FAB) m/z: Calc'd for C₃₁H₆₁N₄O₉P (M+H⁺): 665.4254. Found: 665.4256.

¹H-NMR (CDCl₃: δ 1.42 (s, 18H), 1.51 (s, 18H), 2.50-2.95 (m, 24H).

### Example 10

### Alternative Synthesis of Compound of Example 3 (10-[[Bis(1,1-dimethylethoxy)phosphinyl]methyl]-1,4,7,10-tetraazacyclododecane-1,4,7-triacetic acid 1,7-bis(1,1-dimethylethyl)ester)

This example shows an alternative synthetic route, Scheme 3A, used to make the compound of Example 3 (10-[[Bis(1,1-dimethylethoxy)phosphinyl]methyl]-1,4,7,10-tetraazacyclododecane-1,4,7-triacetic acid 1,7-bis(1,1-dimethylethyl)ester) above.

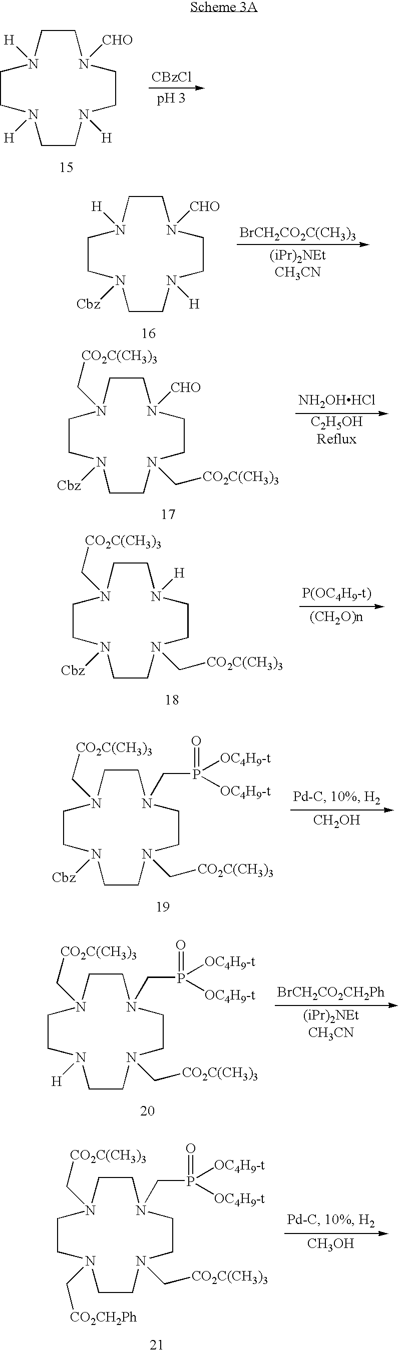

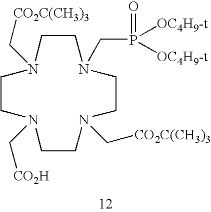

1-Formyl-1,4,7,10-tetraazacyclododecane 15 was selectively protected with benzyl chloroformate at pH 3 to give 16, which was alkylated with t-butyl bromoacetate and deformylated with hydroxylamine hydrochloride to give 18. Reaction with P(OtBu)₃ and paraformaldehyde gave 19, which was deprotected by hydrogenation and alkylated with benzyl bromoacetate to give 21, which was finally hydrogenated to 12.

### Example 10A

This example shows the synthetic route used to make 7-Formyl-1,4,7,10-tetraazacyclododecane-1-carboxylic acid phenyl methyl ester dihydrochloride, (16) in Scheme 3A above.

1-Formyl-1,4,7,10-tetraazacyclododecane 15 (14 g; 69.9 mmol) was dissolved in H₂O (100 mL) and 12 N HCl (11 mL) was added until pH 3, then 1,4-dioxane (220 mL) was added. A solution of benzyl chloroformate (13.8 g; 77 mmol) in 1,4-dioxane (15 mL) was slowly added drop-wise in 3.5 h, constantly maintaining the reaction mixture at pH 3 by continuous addition of 2 N NaOH (68.4 mL) with a pHstat apparatus. At the end of the addition the reaction was stirred for 1 h then washed with n-hexane (4×100 mL) and ⁱPr₂O (4×100 mL). The aqueous phase was brought to pH 13 by addition of 10 N NaOH (6.1 mL) and extracted with CHCl₃ (4×100 mL). The organic phase was washed with brine (100 mL), dried (Na₂SO₄), filtered and evaporated. The oily residue was dissolved in acetone (200 mL) and 6 N HCl (26 mL) was added. The solid precipitated was filtered, washed with acetone (2×50 mL) and dried under vacuum to give compound 16 (23.6 g; 58 mmol) as a white crystalline solid. Yield 83%.

Elemental analysis
for C₁₇H₂₈N₄Cl₂O₃ (%)

| | C | H | N | Cl |
|---|---|---|---|---|
| Calc'd | 50.13 | 6.93 | 13.75 | 17.41 |
| Found | 49.27 | 6.96 | 13.34 | 16.89 |
| Corresp. | 50.29 | 6.87 | 13.62 | 17.24 |

### Example 10B

This example shows the synthetic route used to make 4-(phenylmethoxy)carbonyl-1,4,7,10-tetraazacyclododecane-1,7-diacetic acid bis(1,1-dimethylethyl)ester, (18) in Scheme 3A above.

A solution of 16 (14.4 g; 35.3 mmol) in H₂O (450 mL) and 1 N NaOH (74 mL; 74 mmol) was stirred for 20 min then extracted with CHCl₃ (4×200 mL). The organic layer was evaporated to obtain an oily residue (12.3 g) which was dissolved in CH₃CN (180 mL) and N-ethyldiisopropylamine (DIEA) (15 mL; 88.25 mmol). A solution of t-butyl bromoacetate (16.8 g; 86.1 mmol) in CH₃CN (15 mL) was added drop-wise to the previous solution in 2.5 h. After 20 h at room temperature the solvent was evaporated and the oily residue was dissolved in CHCl₃ (150 mL) and washed with H₂O (5×100 mL). The organic layer was dried (Na₂SO₄), filtered and evaporated to dryness to give 17 as a yellow oil. Crude 17 (22 g) was dissolved in EtOH (250 mL), NH₂OH.HCl (2.93 g; 42.2 mmol) was added and the solution heated to reflux. After 48 h the solvent was evaporated and the residue dissolved in CH₂Cl₂ (250 mL), washed with H₂O (3×250 mL) then with brine (3×250 mL). The organic layer was dried (Na₂SO₄), filtered and evaporated. The oily residue (18.85 g) was purified by flash chromatography. The fractions containing the product were collected and evaporated to obtain a glassy white solid (17.62 g) which was dissolved in H₂O (600 mL) and 1 N NaOH (90 mL; 90 mmol) and extracted with CHCl₃ (3×250 ml). The organic layer was dried (Na₂SO₄) and evaporated to dryness to give 18 (16.6 g; 31 mmol) as an oil (7). Yield 88%.

Elemental analysis for C₂₈H₄₆N₄O₆ (%)

| | C | H | N | Cl | Na | Weight loss % (80° C.) |
|---|---|---|---|---|---|---|
| Calc'd | 62.90 | 8.67 | 10.48 | — | — | |
| Found | 62.47 | 8.86 | 10.35 | 2.02 | 0.21 | 12.82 |

### Example 10C

This example shows the synthetic route used to make 4-(phenylmethoxy)carbonyl-10-[[bis(1,1-dimethylethoxy)phosphinyl]methyl]-1,4,7,10-tetraazacyclododecane-1,7-diacetic acid bis(1,1-dimethylethyl)ester, (19) in Scheme 3A above.

A mixture of compound 18 (13.87 g; 26 mmol), P(OtBu)₃ (7.6 g; 28.6 mmol) (10) and paraformaldehyde (0.9 g; 30 mmol) was heated at 60° C. After 16 h more P(OtBu)₃ (1 g; 3.76 mmol) and paraformaldehyde (0.1 g; 3.33 mmol) were added. The reaction was heated at 60° C. for more 20 h then at 80° C. for 8 h under vacuum to eliminate the volatile impurities. The crude was purified by flash chromatography to give 19 (9.33 g; 8 mmol) as an oil. Yield 31%.

### Example 10D

This example shows the synthetic route used to make 7-[[bis(1,1-dimethylethoxy)phosphinyl]methyl]-1,4,7,10-tetraazacyclododecane-1,4,10-triacetic acid 1-phenylmethyl 4,10-bis(1,1-dimethylethyl)ester, (21) in Scheme 3A above.

To the solution of 19 (6.5 g; 5.53 mmol) in CH₃OH (160 mL) 5% Pd/C (1 g; 0.52 mmol) was added and the mixture was stirred under hydrogen atmosphere at room temperature. After 4 h (consumed H₂ 165 mL; 6.7 mmol) the mixture was filtered through a Millipore® filter (FT 0.45 μm) and the solution evaporated under reduced pressure. The crude (5.9 g) was purified by flash chromatography to give 20 (4.2 g) as an oil. Benzyl bromoacetate (1.9 g; 8.3 mmol) dissolved in CH₃CN (8 mL) was added drop-wise in 1 h to a solution of 20 (4.2 g) in CH₃CN (40 mL) and DIEA (1.5 mL; 8.72 mmol). After 36 h at room temperature the solvent was evaporated and the residue (5.76 g) dissolved in CHCl₃ (100 mL), washed with H₂O (2×100 mL) then with brine (2×70 mL). The organic layer was dried (Na₂SO₄), filtered and evaporated. The crude (5.5 g) was purified twice by flash chromatography, the fractions were collected and evaporated to dryness to afford 21 (1.12 g; 1.48 mmol) as an oil. Yield 27%.

### Example 10E

This example shows the synthetic route used to make 7-[[Bis(1,1-dimethylethoxy)phosphinyl]methyl]-1,4,7,10-tetraazacyclododecane-1,4,10-triacetic acid 4,10-bis(1,1-dimethylethyl)ester, (12) in Scheme 3A.

5% Pd/C (0.2 g; 0.087 mmol) was added to a solution of 21 (1.12 g; 1.48 mmol) in CH₃OH (27 mL) and the mixture was stirred under hydrogen atmosphere at room temperature. After 2 h (consumed H₂ 35 mL; 1.43 mmol) the mixture was filtered through a Millipore® filter (FT 0.45 μm) and the solution evaporated to dryness to give 12 (0.94 g; 1.41 mmol) as a pale yellow oil. Yield 97%.

### Example 11

### Synthesis of the Compound of Example 4 (10-[[Bis(1,1-dimethylethoxy)phosphinyl]methyl]-α′-(carboxymethyl)-1,4,7,10-tetraazacyclododecane-1,4,7-triacetic acid α,α′,α″-tris(1,1-dimethylethyl)ester)

This example shows the synthetic route used to make the compound of Example 4 above.

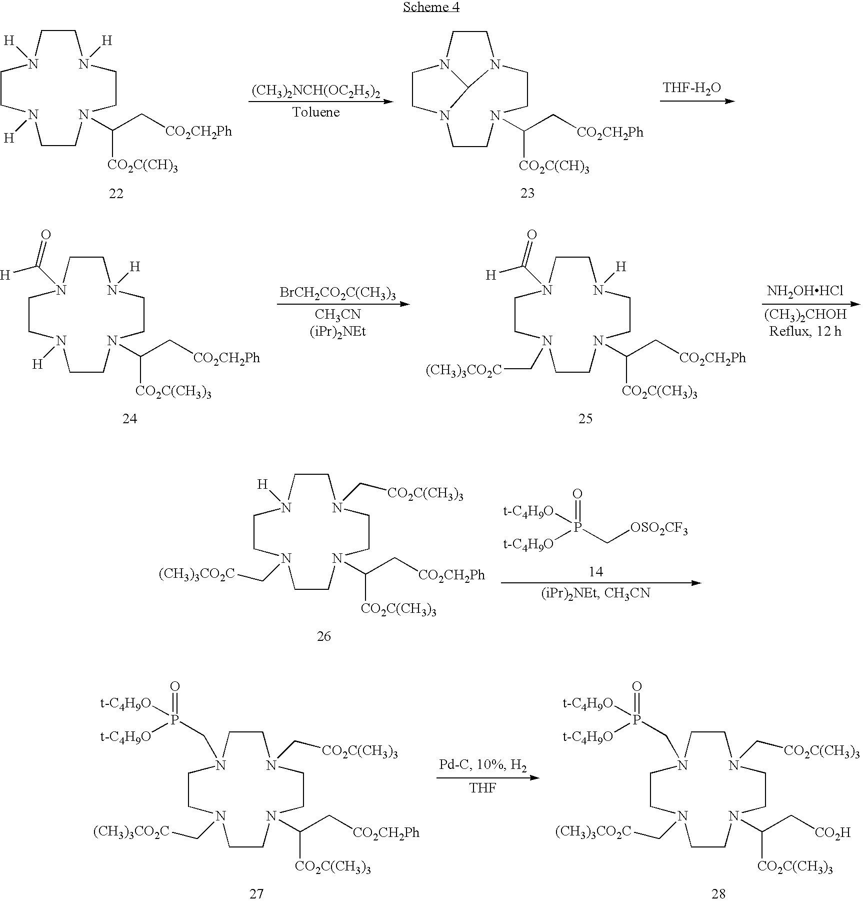

Compound 22 was reacted with N,N-dimethylformamide diethyl acetal in toluene to give the tricycle 23 which was treated with water-THF mixture for 24 h to give the formyl derivative 24. Alkylation of the formyl compound with tert-butyl bromoacetate followed by purification gave the fully protected macrocycle 25 in 50% yield.

Deformylation of 25 using hydroxylamine hydrochloride in isopropanol gave tritertiarybutyl ester 26 in 78% yield. Alkylation of tritertiarybutyl ester 26 with triflate A in acetonitrile in the presence of diisopropylethylamine afforded 27 was in 56% yield. Debenzylation of 27 using Pd—C 10% in THF gave the acid 28.

### Example 11A

This example shows the synthetic route used to make octahydro-7H,9bH-2a,4a,7,9a-tetraazacycloocta[cd]pentalene-7-acetic acid α-[[(phenylmethoxy)carbonyl]methyl]1,1-dimethylethyl ester, (23) in Scheme 4 above.

To a solution of 1,4,7,10-tetraazacyclododec-1-yl-butanedioic acid 1-(1,1-dimethylethyl)4-(phenylmethyl)ester (22) (3.0 g; 6.9 mmol) in benzene (25 mL) was added dimethylformamide diethyl acetal (1.2 g; 1.4 mL, 8.0 mmol) and the mixture was heated under reflux at 110° C. for 12 h. The ethanol and the diethylamine formed were removed by azeotropic distillation using a Dean Stark water separator. The solvent was removed and the residue was dried under vacuum for 3 h. The octahydro-7H,9bH-2a,4a,7,9a-tetraazacycloocta[cd]pentalene-7-acetic acid α-[[(phenylmethoxy)carbonyl]methyl]1,1-dimethylethyl ester (23) obtained was used as such in the next step without further purification. Yield 2.98 g (97%).

MS: (M+H)⁺=445.

HRMS (FAB) m/z: Calc'd for C₂₄H₃₆N₄O₄ (MH⁺): 445.2815. Found: 445.2822.

¹H-NMR (CDCl₃): δ 1.42 (s, 9H), 2.45 and 2.55 (2×m, 2H), 2.65-3.05 (m, 16H), 3.82 (s, 1H), 4.65 (s, 1H), 5.12 (quart, 2H), 5.12 (s, 2H), 7.38 (m, 5H).

### Example 11B

This example shows the synthetic route used to make 7-Formyl-1,4,7,10-tetraazacyclododecane-1-acetic acid acid α-[[(phenylmethoxy)carbonyl]methyl]1,1 dimethylethyl ester, (24) in Scheme 4 above.

Octahydro-7H,9bH-2a,4a,7,9a-tetraazacycloocta[cd]pentalene-7-acetic acid α-[[(phenylmethoxy)carbonyl]methyl]1,1-dimethylethyl ester (23) (2.9 g; 6.53 mmol) was added to a mixture of THF-water (1: 1, 30 mL) and the solution was stirred at room temperature for 12 h. THF-water were removed on a rotary evaporator and the oil obtained was dried under vacuum for 4 h. The formyl derivative 24 thus obtained was used in the next step without purification. Yield 2.92 g (96%).

¹H-NMR (CDCl₃): δ 1.42 (s, 9H), 2.45-3.05 (m, 19H), 3.73 (m, 1H), 4.65 (s, 1H), 5.12 (m, 2H), 7.38 (m, 5H), 8.12 (s).

### Example 11C

This example shows the synthetic route used to make α′-[[(phenylmethoxy)carbonyl]methyl]-10-formyl-1,4,7,10-tetraazacyclododecane-1,4,7-triacetic acid acid tris(1,1dimethylethyl)ester, (25) in Scheme 4 above.

To a mixture of α-[[(phenylmethoxy)carbonyl]methyl]-7-formyl-1,4,7,10-tetraazacyclododecane-1-acetic acid acid 1,1 dimethylethyl ester 24 (3.0 g; 6.5 mmol) and N,N-diisopropylethylamine (2.26 g; 3.1 mL, 17.5 mmol) in acetonitrile was added tert-butyl bromoacetate (3.12 g; 2.4 mL, 16.4 mol) and the mixture was stirred at room temperature for 12 h. Acetonitrile was removed and the residue was treated with sodium bicarbonate solution (15 mL). The mixture was then extracted with ethyl acetate, washed with water (2×50 mL), sodium chloride solution (50 mL) and dried Na₂SO₄. The ethyl acetate layer was concentrated to give an oil, which was purified by silica gel column chromatography using hexane-ethyl acetate (50:50, 25: 75, 100). Fractions containing the product were collected and the solvent evaporated to give α′-[[(phenylmethoxy)carbonyl]methyl]-10-formyl-1,4,7,10-tetraazacyclododecane-1,4,7-triacetic acid acid tris(1,1dimethylethyl)ester (25) as a thick oil. Yield 2.5 g (56%).

MS: (M+H)⁺=691.

HRMS (FAB) m/z: Calc'd for C₃₆H₅₈N₄O₉ (MH⁺): 691.4282. Found: 691.4315.

¹H-NMR (CDCl₃): δ 1.42 (2 s, 27H), 2.52-2.58 (m, 16H), 3.20 and 3.50 (2×s and m, 5H), 3.30 and 3.62 (2×m, 2H), 3.78 (m, 1H), 5.05 (s, 2H), 7.30 (m, 5H), 7.98 (s, 1H).

### Example 11D

This example shows the synthetic route used to make α′-[[(phenylmethoxy)carbonyl]methyl]-1,4,7,10-tetraazacyclododecane-1,4,7-triacetic acid acid tris(1,1dimethylethyl)ester, (26) in Scheme 4 above.

To a solution of the α′-[[(phenylmethoxy)carbonyl]methyl]-10-formyl-1,4,7,10-tetraazacyclododecane-1,4,7-triacetic acid acid tris(1,1dimethylethyl)ester 25 (2.46 g; 3.56 mmol) in isopropanol (15.0 mL) was added hydroxylamine hydrochloride (0.312 g; 4.5 mmol) and the mixture was heated at 90° C. for 12 h. The reaction was followed by TLC (CH₂Cl₂:CH₃OH, 95:5). The solvent was removed on a rotary evaporator and the residue was treated with a saturated solution of sodium bicarbonate solution (10 mL) and extracted with ethyl acetate. The ethyl acetate solution was washed with water and dried (Na₂SO₄). This product 26 was used in the next step with out further purification. Yield 2.32 g (98%).

MS: (M+H)⁺=663.5, 691.5 (unreacted starting material)

¹H-NMR (CDCl₃): δ 1.42 (3 s, 27H), 2.52-3.05 (m, 18H) 3.31 (m, 4H), 3.89 (m, 1H), 5.15 (m, 2H), 7.30 (m, 5H).

### Example 11E

This example shows the synthetic route used to make 10-[[bis(1,1-dimethylethoxy)phosphinyl]methyl]-α′-[[(phenylmethoxy)carbonyl]methyl]-1,4,7,10-tetraazacyclododecane-1,4,7-triacetic acid acid tris(1,1dimethylethyl)ester, (27) in Scheme 4 above.

To a mixture of α′-[[(phenylmethoxy)carbonyl]methyl]-1,4,7,10-tetraazacyclododecane-1,4,7-triacetic acid acid tris(1,1dimethylethyl)ester (26) (2.2 g; 3.32 mmol) and N,N-diisopropylethylamine (2.6 g; 3.6 mL, 20 mmol) in acetonitrile (10 mL) was added di-tert-butyl phosphonomethyltriflate (14) (Phillion, D. P., U.S. Pat. No. 4,740,608 (1988)) (2.5 g; 7.0 mmol) in acetonitrile (5.0 mL) and the mixture was stirred at 40° C. for 6 h. After the reaction acetonitrile was removed and the residue was treated with sodium bicarbonate solution (25 mL). The mixture was then extracted with ethyl acetate (2×50 mL) washed with water (50 mL) and dried (Na₂SO₄). Ethyl acetate was removed to give an oil, which was chromatographed over silica gel using methylene chloride and methanol (9:1) containing 0.1% triethylamine. Fractions containing the product were collected and evaporated to give an oil, which was dried, under vacuum to give 27 as a foamy solid. Yield 1.4 g (49%).

MS: (M+H)⁺=869.6.

HRMS (FAB) m/z: Calc'd for C₄₄H₇₇N₄O₁₁P (M+Na): 891.5224. Found: 891.5233.

¹H-NMR (CDCl₃): δ 1.38-1.52 (5 s, 45H), 2.05-3.60 (m, 24H), 4.10 (m, 1H), 5.05 (quart, 2H), 7.30 (m, 5H).

### Example 11F

This example shows the synthetic route used to make 10-[[bis(1,1-dimethylethoxy)phosphinyl]methyl]-α′-(carboxymethyl)-1,4,7,10-tetraazacyclododecane-1,4,7-triacetic acid acid tris(1,1dimethylethyl)ester, (28) in Scheme 4 above.

To a solution of 10-[[bis(1,1-dimethylethoxy)phosphinyl]methyl]-α′-[[(phenylmethoxy)carbonyl]methyl]-1,4,7,10-tetraazacyclododecane-1,4,7-triacetic acid acid tris(1,1dimethylethyl)ester (27) (1.18 g; 1.35 mmol) in THF (15 mL) was added Pd—C 10% (0.4 g, Degussa type ˜50% water) and the mixture was hydrogenated at 45 psi for 6 h. The catalyst was filtered through celite and the filter cake was washed with THF (2×30 mL). The combined THF solution was concentrated on a rotary evaporator to give the acid as a thick viscous oil. This was dried under vacuum for 24 h to give 28 as a foamy solid and used as such without further purification. Yield. 0.96 g (95%).

MS: (M+H)⁺=779.6.

HRMS (FAB) m/z: Calc'd for C₃₇H₇₁N₄O₁₁P (M+Na): 801.4755. Found: 801.4805.

¹H-NMR (CDCl₃): δ 1.42-1.52 (5 s, 45H), 2.05-3.72 (m, 24H), 4.0 (m, 1H).

### Example 12

### Synthesis of the Compound of Example 5 (10-[[1-[Bis(1,1-dimethylethoxy)phosphinyl]-3-carboxy]propyl]-1,4,7,10-tetraazacyclododecane-1,4,7-triacetic α,α′,α″-tris(1,1-dimethylethyl)ester)

Scheme 5 shows the synthetic route used to make the compound of Example 5 above.

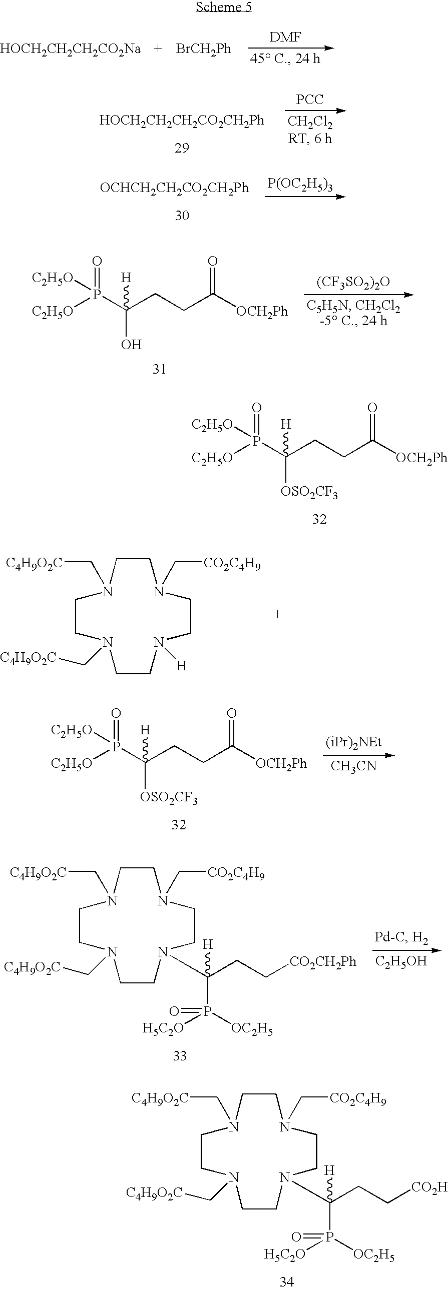

Benzyl 4-hydroxybutyrate was prepared by selective benzylation of 4-hydroxybutyric acid sodium salt with benzyl bromide. Oxidation of 29 with pyridinium chlorochromate afforded the aldehyde 30. Successive treatment of the aldehyde, first with triethylphosphite and with triflic anhydride in the presence of diisopropylethylamine furnished the trifluoromethanesulfonyloxy derivative 32. Alkyation of DO3A-tri-t-butylester by the triflate 32 afforded the benzyl ester 5 in 45% yield. Hydrogenation of 33 with Pd—C in ethanol afforded the acid 34.

### Example 12A

This example shows the synthetic route used to make 10-[[1-[bis(1,1-dimethylethoxy)phosphinyl]-3-[(phenylmethoxy)carbonyl]]propyl]-1,4,7,10-tetraazacyclododecane-1,4,7-triacetic α,α′,α″-tris(1,1-dimethylethyl)ester, (33) in Scheme 5 above.

A solution of the triflate 31 4.62 g (0.01 mol) in acetonitrile (15 mL) was added drop-wise to a mixture of DO3A-t-butylester 5.14 g (0.01 mol) and diisopropylethylamine 2.6 g (3.6 mL, O₂ mol) in dry acetonitrile (25 mL) over a period of 45 min. Exothermic reaction was observed during the addition and the mixture was stirred at room temperature for 12 h. Acetonitrile was then removed on a rotary evaporator and the residue was treated with water and extracted with ethyl acetate. The ethyl acetate layer was washed with water and dried (Na₂SO₄). Evaporation of ethyl acetate gave a thick viscous oil which was chromatographed over silica gel. The column was prepared with methylene chloride and ether (95:5) and elution with methylene chloride and ether gave a fast moving UV visible impurity. The NMR spectrum of the fast moving product suggested that it is the elimination product. The column was then eluted with methylene chloride and methanol (9:1). The product containing fractions were collected and evaporated to give a thick oil, which was dried, under vacuum to give a foamy solid. Yield: 2.52 g (30%).

TLC: CH₂Cl₂:CH₃OH, 9:1 (Rf 0.8).

MS: (M+H)=827

¹HNMR (CDCl₃): δ 1.18 and 2.28 (2×t, 6H), 1.35 and 1.42 (3×s, 27H) 1.8-2.2 (m 4H), 2.2-3.3 (m, 16H), 3.38-3.34 (m, 6H), 3.95-4.15 (m, 4H), 4.30 (m, 1H), 5.05 (s, 2H), 7.35 (m, 5H).

### Example 12B

This example shows the synthetic route used to make 10-[[1-[bis(1,1-dimethyl ethoxy)phosphinyl]-3-carboxy]propyl]-1,4,7,10-tetraazacyclododecane-1,4,7-triacetic α,α′,α″-tris(1,1-dimethylethyl)ester (34) in Scheme 5 above.

To a solution of 10-[[1-[bis(1,1-dimethylethoxy)phosphinyl]-3-[(phenylmethoxy)carbonyl]]propyl]-1,4,7,10-tetraazacyclododecane-1,4,7-triacetic α,α′,α″-tris(1,1-dimethylethyl)ester 3.0 g (0.0036 mol) in ethanol (50 mL) was added Pd—C 10% (2.0 g) and the mixture was hydrogenated at 45 psi. The catalyst was removed and ethanol was evaporated to give the acid 34 as a foamy solid. Yield 2.51 g (95%).

MS: (M+H)=737

¹HNMR (CDCl₃): δ 18 and 2.20 (2×t, 6H), 1.45 (3 s, 27H) 1.8-2.2 (m 4H), 2.2-3.3 (m, 16H), 3.38-3.34 (m, 6H), 3.95-4.15 (m, 4H), 4.40 (m, 1H).

### Example 13

### Synthesis of the Compound of Example 6 (4,10-Bis[[bis(1,1-dimethylethoxy)phosphinyl]methyl]-1,4,7,10-tetraazacyclododecane-1,7-diacetic (1,1-dimethylethyl)ester)

Scheme 6 shows the synthetic route used to make the compound of Example 6 (a bisphosphonic acid analog containing a carboxylic group) above.

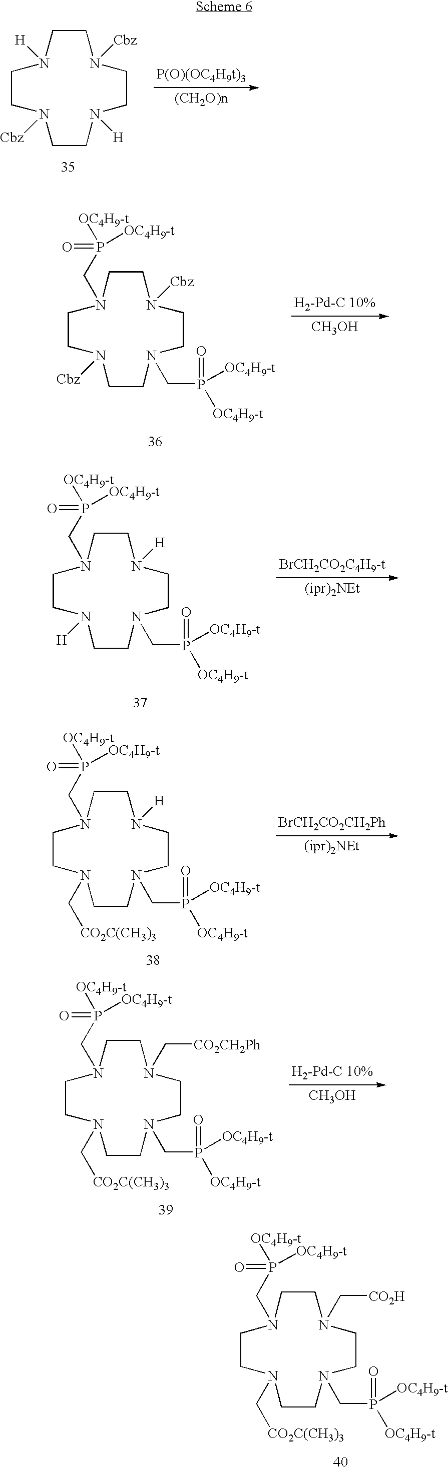

Treatment of compound 35 with tri t-butyl phosphate and paraformaldehyde is expected to provide the orthogonally protected ligand 36. Catalytic hydrogenolysis of 36 is expected to afford compound 37. Alkylation with t-butylbromo acetate in the presence of diisopropylethylamine is expected to furnish compound 38. Alkylation of 38 with benzyl-2-bromoacetate followed by debenzylation employing Pd—C is expected to afford compound 40.

### Example 14

This example shows a process for conjugating a compound of this invention to a peptide.

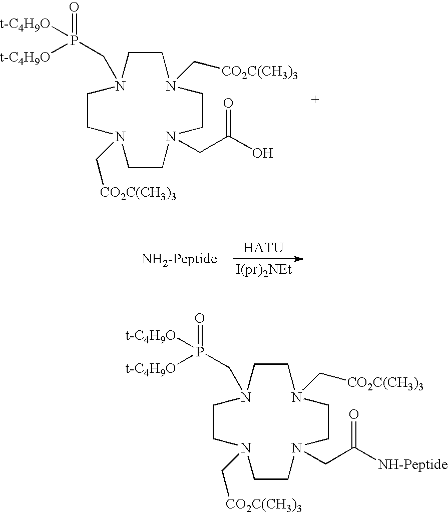

It will be understood by a person skilled in the art that other peptide coupling reagents, such as DCC, HOBT and others, can be used instead of the HATU shown above. Such conjugation to peptides and other linkers and targeting moieties can be accomplished using, for examples, carboxyl and/or amino groups, if present, on the compounds of this invention.

### Example 15

As stated above, the compounds of this invention may also be combined to form homo and hetero dimers and homo and hetero multimers. As example of a homo dimer and a process of making it is shown below:

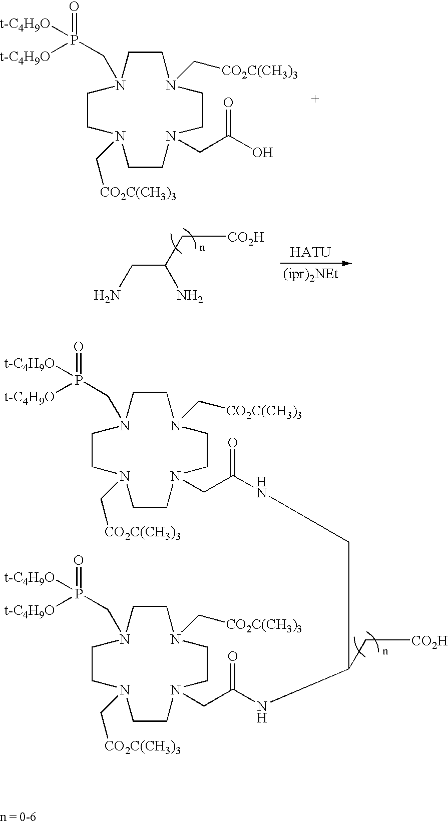

Preferred homo and hetero dimers are comprised of the compounds of Examples 1-6. Preferred homo and hetero multimers are also comprised of the compounds of Examples 1-6.

Throughout the foregoing description of the invention, various patents, articles, and other publications have been cited or referenced. The entire contents of each patent, article and other publication is hereby incorporated by reference into the subject application.

1-23. (canceled)

24. A compound comprising a polyazamacrocyclic compound and at least one phosphonic group substituted on at least one of the aza groups of said polyazamacrocyclic compound or a salt form thereof.

25. The compound of claim 24 wherein said polyazamacrocyclic compound comprises the general formula (II): 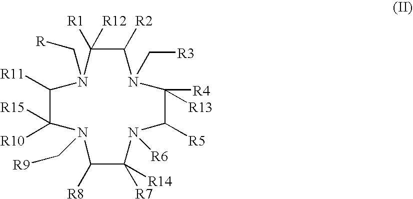
where
R¹═R²═R⁷═R⁸═R¹⁰═R¹¹═H;
R¹², R¹³, R¹⁴, and R¹⁵═CH₃ or H;
R⁴═R⁵; and R¹⁰═R¹¹ can be H or groups taken together forming a cyclic C₃-C₄ alkene group;
R⁶═X, where X═CH₂P(O)(OH)₂, CH₂P(O) (OC₄H₉-t)₂, CH₃CHP(O) (OH)₂, CHP(O) (OH)₂—(CH₂)ₙCO₂H, CHP(O) (OH)₂, (CH₂)ₙNH₂, CHP(O)(OH)₂-Aryl-CO₂H, CHP(O) (OH)₂-Aryl-NH₂ or CHP(O)(OH)₂-Aryl-NHCS, where n=1-12; and
R, R³, or R⁹ is CO₂C(CH)₃, or CO₂H;
or a salt form thereof.

26. The compound of claim 24 wherein said polyazamacrocyclic compound comprises the general formula (IV): 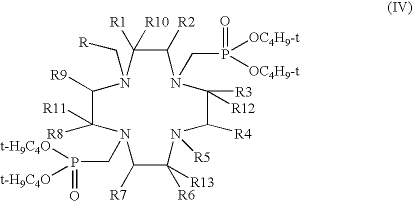
where
R¹═R²═R³═R⁴═R⁶═R⁷═R⁸R⁹═H;
R³═R⁴ and R⁸═R⁹ can be H or groups taken together forming a cyclic C₃-C₄ alkene group;
R¹⁰, R¹¹, R¹² and R¹³═CH₃ or H;
R═CO₂C(CH₃)₃; and
R⁵═CH₂P(O)(OH)₂, CH₂P(O)(OC₄H₉-t)₂, CH₃CHP(O)(OH)₂, CHP (O)(OH)₂—(CH₂)ₙCO₂H, CHP(O)(OH)₂, (CH₂)ₙNH₂, CHP(O) (OH)₂-Aryl-CO₂H, CHP(O)(OH)₂-Aryl-NH₂ or CHP(O)(OH)₂-Aryl-NHCS, where n=1-12;
or a salt form thereof.

27. A compound of the formula:
10-Phosphonomethyl-1,4,7,10-tetraazacyclododecane-1,4,7-triacetic acid (MPDO3A);
10-(1-phosphonomethyl)-1,4,7,10-tetrazacycododecane-1,4,7-triacetic acid;
10-[[Bis(1,1-dimethylethoxy)phosphinyl]methyl]-1,4,7,10-tetraazacyclododecane-1,4,7-triacetic acid 1,7-bis(1,1-dimethylethyl)ester;
10-[[Bis(1,1-dimethylethoxy)phosphinyl]methyl]-α′-(carboxymethyl)-1,4,7,10-tetraazacyclododecane-1,4,7-triacetic acid α,α′,α″-tris(1,1-dimethylethyl)ester;
10-[[1-[Bis(1,1-dimethylethoxy)phosphinyl]-3-carboxy]propyl]-1,4,7,10-tetraazacyclododecane-1,4,7-triacetic α,α′,α″-tris(1,1-dimethylethyl)ester; or
4,10-Bis[[bis(1,1-dimethylethoxy)phosphinyl]methyl]-1,4,7,10-tetraazacyclododecane-1,7-diacetic (1,1-dimethylethyl)ester;
or a salt form thereof.

28. A compound comprising a homo dimer, hetero dimer, homo multimer or hetero multimer of the compound of any of claims 24-27.

29. A complex comprising the compound of any of claims 24-27 complexed with a paramagnetic or radionuclide metal or a salt form thereof.

30. A method for preparing a complex comprising the step of conjugating the compound of any of claims 24-27 with a paramagnetic or radionuclide metal.

31. A method of imaging comprising the steps of:
administering to a patient a diagnostic imaging agent comprising the compound of any of claims 24-27 complexed with a paramagnetic or radionuclide metal, and
imaging said patient.

32. A method for preparing a diagnostic imaging agent comprising the step of adding to an injectable medium a substance comprising the compound of any of claims 24-27.

33. A kit for preparing a diagnostic imaging agent comprising the compound of any of claims 24-27.

34. A kit for preparing a radiotherapeutic agent comprising the compound of any of claims 24-27.

35. A method of treating a patient comprising the step of administering to a patient a radiotherapeutic agent comprising the compound of any of claims 24-27 complexed with a therapeutic radionuclide.

36. A method of preparing a radiotherapeutic agent comprising the step of adding to an injectable therapeutic medium a substance comprising at least one compound of any of claims 24-27.

37. The compound of any of claims 24-27 further comprising a linking group, a targeting moiety, a diagnostic moiety and/or a therapeutic moiety.

38. The complex of claim 29 further comprising a linking group, a targeting moiety, a diagnostic moiety and/or a therapeutic moiety.

39. A method for preparing a polyazamacrocyclic compound bound to a linker, targeting moiety, diagnostic moiety or therapeutic moiety comprising the step of:
conjugating a polyazamacrocyclic compound to a linker, targeting moiety, diagnostic moiety or therapeutic moiety with a coupling agent, wherein:
said coupling agent is selected from the group consisting of DCC, HOBT and HATU,
said polyazamacrocyclic compound comprises one carboxyl group and/or at least one amino group, and
said linker, targeting moiety, diagnostic moiety or therapeutic moiety comprises at least one amino or acid functional group.


SEQUENCE LISTING (4 sequences)

SEQ ID NO: 1 - protein, 8 aa, Artificial
  Description of Artificial Sequence analogs of somatostatin
ACTWKVCT

SEQ ID NO: 2 - protein, 8 aa, Artificial
  Description of Artificial Sequence analogs of somatostatin
ACTWKVCT

SEQ ID NO: 3 - protein, 8 aa, Artificial
  Description of Artificial Sequence analogs of somatostatin
FCYWKTCT

SEQ ID NO: 4 - protein, 10 aa, Artificial
  Description of Artificial Sequence LHRH analog
EHWXYKXXPX